SEQUENCE LISTING (54 sequences)

SEQ ID NO: 1 - protein, 563 aa, Cocos nucifera
MAGYSVAAPLCTWLVAACVTASGGKEGSLVAPAVGEARRLSRSARRRRAAALRVEARDSSGGLMSALRGSGIQGLMSSCLAFEPCAEFYGSKGASAFFGESGFSLFGTWKAETTRRQRRAARASCVSGKAMAIAVQPAKEIAEKKRIHMKKRRVVVTGMGVVTPLGDDPDIFYNNLLDGVSGISQIETFDCTNFPTRIAGEIKSFSTDGLVAPKLSKRMDKFMLYLLIAGKKALANGGVTEEVMSQLDKAKCGVLIGSAMGGMKVFNDAIEALRVSYKKMNPFCVPFATTNMGSAILAMDLGWMGPNYSISTACATSNFCILNAAHHIIRGEADAMLCGGSDATIIPIGLGGFVACRALSQRNSDPTKASRPWDIDRDGFVMGEGAGVLLLEELEHAKQRGANIYAEFLGGSFTCDAYHMTEPHPEGAGIALCIENALAQAGVAKEDVNYVNAHATSTPAGDLKEYQALIRCFGQNPELRVNSTKSMIGHLLGAAGAVEAVASIQAIRTGWVHPNINLENPEKSVDINVLVGSRKERLDVKVALSNSFGFGGHNSSILFAPYK

SEQ ID NO: 2 - DNA, 1692 bp, Cocos nucifera
atggccgggtactcggtggcggcgccgctgtgcacttggttggtggcggcgtgcgtcacggcgtcgggcggaaaggaggggtctttggtggcgccggcggtcggggaggcgaggcggttgagccggtcggcgaggaggcggagggcggcggcgctacgagtcgaagcccgggattcctctgggggactgatgtcggcgctccgtggatcggggatccaggggctgatgagctcctgcctcgccttcgagccctgcgcggagttctacggctctaagggcgcgtcggcgttcttcggggagagtggcttctctctctttgggacgtggaaggcggagactacaagaaggcagcgaagggccgcgcgcgcctcttgcgtctcaggcaaagcaatggcaatagctgtgcagcctgctaaggaaattgcagaaaagaagagaatccatatgaagaagaggagagtggtcgtgacagggatgggtgtggtgactccactgggcgatgatcctgatatcttctacaataaccttcttgatggtgtcagtggtataagtcaaattgaaacatttgactgtacaaactttccaacaagaattgcaggagaaattaaatctttctcaacagatggattggtggcacctaaattatctaaacgaatggacaaattcatgctctatttacttattgctggaaagaaagcattagccaatggtggggttactgaagaggtcatgagtcagcttgacaaggcaaaatgcggagtgctcataggctctgcaatgggtggaatgaaggtttttaatgatgccatcgaagctttaagggtctcatataagaagatgaatccattttgtgttccatttgcaacaacaaacatgggttctgcaatccttgctatggatctgggttggatgggcccaaattactctatttcaactgcttgtgctacaagcaatttctgtatcctgaatgcagcacaccatataataagaggggaagctgatgcaatgctttgtggtggatcagatgctacaattataccgattggattgggggggtttgttgcttgcagagcactttcgcagagaaatagtgatccgactaaagcatcgcggccttgggacattgatcgtgatggatttgtgatgggggaaggggctggtgtgcttctactggaagaattagagcatgctaagcaaagaggagctaatatctatgctgaatttcttggaggaagcttcacgtgtgatgcttaccacatgactgagccacatcctgagggggcaggcattgctctttgcattgagaatgcattagcacaagctggggtagccaaagaagatgttaattatgtaaatgctcatgcaacttcaacacctgctggtgatctaaaagagtatcaagctcttattcgttgttttgggcagaatcctgagctgagagtgaactctacaaaatccatgattggtcacctactaggagctgctggtgcagtggaagctgttgcttcaattcaggcaattcgaacagggtgggtccatcccaatatcaatctcgaaaacccagaaaaaagtgtggatataaatgtgctggtgggctcgagaaaggaaaggttggatgtgaaggtggcattatcaaactcattcggttttggtggccacaactcgtctatcttgtttgcgccttacaaatag

SEQ ID NO: 3 - DNA, 1485 bp, Cocos nucifera
atggccacaagtgctagtatcggggtcgcgggaaaggagttgggagtgatcagcgaagcctcatctcctctcaagcggtataatggcctaaggcctttgctggggagcaagcagatggcttcctttgctgctatgaggaaaccaaatggtccctttccttctttgccatctcttaaatctccaaggataagagctgtggcttccccaactgttgctgctccaaagcgagagaaggatcccaagaaaaggatagtggtgactgggataggcctggtctcggtgtttgggagcgacattgatacattctacaacaagctcttggaaggacagagcgggattagcctgatcgaccgatttgatgcttcttcttactctgtccgattcggtggacagatccgggacttctcctcgaaaggctacattgatgggaagaatgatcgccggctcgatgactgctggagatattgcctggtcgccggcaaaagagctcttgatgatgctaatctcggaccagaagtcctgcaatctatggacaggtcgagaattggagtgctggtagggacaggcatgggtggtttaacggctttcagcaatggagttgaggctctgatccaaaagggttacaagaaaattactcctttcttcattccttactccatcacaaacatgggatcggcgttgttagctatagaaacaggcttaatgggaccaaactactccatttccacggcatgtgcaactgcaaactactgcttttatgctgctgccaatcacataaggagaggtgaagctgacattatggttgctggaggaacagaggcggcaatcctgcctactggagttggtggattcatcgcatgcagagcactgtcacaaagaaatgatgaaccacagaaagcttcgaggccttgggacaaagaccgagatggtttcgtcatgggagagggatctggtgtcctcattatggagagtctagagcatgcaagaaagaggggtgcaactataattgcagagtatcttggaggtgccataacctgtgatgcgcatcacatgaccgatcctcgttctgatggacttggagtctcttcttgcattgttaagagcttggaagatgcaggagtctcccccgaggaggtgaattatgtcaatgctcatgcaacatcaacacttgctggagatttagcagaagttaatgccatcaagaaggttttcaaagacacatccgaaatgaaaatgaatggaacaaagtcgatgattgggcattgccttggagctgctggtgggctggaagcaattgcaaccatcaaagctatcacaacaggctggctgcatccaaccatcaaccaaaataacttggagcctgatgtcaccgtcgacaccatccccaatgtaaagaagaagcatgaggttaatgttgccatctttaattcgtttggtttcgggggtcacaattctgtggttgtttttgctcccttcatgccttaa

SEQ ID NO: 4 - DNA, 1413 bp, Cocos nucifera
atggacggcctccgccttccctccatggacggcctccgcctctccccctccccaccgccggccgccgccccctcccggcgctgccttcttctccgtcgatccgtccggcggcccgccgtccgcgcctccgtcgcggtggcgccccggagggagacggatccgaagaagcgggttgtgataacggggatggggctggtgtcggtgttcgggaacgacgtggatgtttattacaagaagctgcttgaaggggagagcgggatcgggccgatcgaccggtttgacgcctctaagttcccgacgaagttcgctggccagatcaggggtttctcgtcggagggatacatcgacgggaagaacgaccggcggcttgacgactgcctcaggtactgcctcgtcagtggcaagaaggcactggagagcgccggacttggcgttgggagcaaggatctcagcaagattgacaaggtgcgagctggtgtacttgtggggacaggcatgggtggcctcacggtgttttctgatggtgtccaggctctcatagagaaaggccacaggaaaataactccattttttattccttatgccataacaaacatgggctccgccttgctggcaatggatattggcttcatgggtgcaaattattcaatttcaactgcctgtgctacttcaaattattgtttctatgctgctgccaaccatattcgtcgtggtgaggctgatgtaatgattgctggtggtaccgaagctgcaattattccaattggggttggggggtttgtggcctgtagggcattgtcgcagagaaatgatgatcccaaaactgcatcaaggccatgggacaaaggtcgggatggctttgttatgggagaaggtgctggagtgttggtcatggagagtctggaacatgcaatgaaacgtgatgcacctattattgctgaatacttgggaggtgctgtcaactgcgatgcttatcatatgacagatcctagagctgacgggcttggcgtttcatcttgtatcgaaaaaagccttgaagatgccggagttgcaccagaagaggttaactatataaatgcccatgcgacttccacacttgctggtgacctagctgaggtgaatgctgttaaacaggtcttcaagaacccatcagaaatcaagatgaatgcaaccaagtctatgatagggcactgccttggtgcagctggaggtttagaagccattgcaacagtgaaagccataactactgggtggctgcatccaaccataaaccaatttaatccagagcctgcagttgaattcgatacagtggcaaataaaaagcagcaacatgaagtgaatgttgccatttctaattcatttggatttggaggacacaactctgtggtggtatttgcaccttctaagccatga

SEQ ID NO: 5 - protein, 303 aa, Cocos nucifera
LDSKKRGADAVADASGVGKMVKNGLVYRQNFSIRSYEIGVDKRASVEALMNHFQETSLNHCKCIGLMHGGFGCTPEMTRRNLIWVVAKMLVHVERYPWWGDVVQINTWISSSGKNGMGRDWHVHDCQTGLPIMRGTSVWVMMDKHTRRLSKLPEEVRAEITPFFSERDAVLDDNGRKLPKFDDDSAAHVRRGLTPRWHDFDVNQHVNNVKYVGWILESVPVWMLDGYEVATMSLEYRRECRMDSVVQSLTAVSSDHADGSPIVCQHLLRLEDGTEIVRGQTEWRPKQQACDLGNMGLHPTESK

SEQ ID NO: 6 - DNA, 912 bp, Cocos nucifera
ctcgattccaagaagaggggggccgacgcggtcgcagatgcctctggggtcgggaagatggtcaagaatggacttgtttacaggcagaatttttctatccggtcctacgaaatcggggttgataaacgtgcttcggtagaggcattgatgaatcatttccaggaaacgtcgcttaaccattgcaagtgtattggccttatgcatggcggctttggttgtacaccagagatgactcgaagaaatctgatatgggttgttgccaaaatgctggttcatgtcgaacgttatccttggtggggagacgtggttcaaataaatacgtggattagttcatctggaaagaatggtatgggacgtgattggcatgttcatgactgccaaactggcctacctattatgaggggtaccagtgtctgggtcatgatggataaacacacgaggagactgtctaaacttcctgaagaagttagagcagagataacccctttcttttcagagcgtgatgctgttttggacgataacggcagaaaacttcccaagttcgatgatgattctgcagctcatgttcgaaggggcttgactcctcgttggcatgatttcgatgtaaatcagcatgtgaacaatgtcaaatacgtcggctggattcttgagagcgttcctgtgtggatgttggatggctacgaggttgcaaccatgagtctggaataccggagggagtgtaggatggatagtgtggtgcagtctctcaccgccgtctcttccgaccacgccgacggctcccccatcgtgtgccagcatcttctgcggctcgaggatgggactgagattgtgaggggtcaaacagaatggaggcctaagcagcaggcttgtgatcttgggaacatgggtctgcacccaactgagagtaaatga

SEQ ID NO: 7 - protein, 287 aa, Nannochloropsis oculata NIES2145
MTPLAFTVLGKLGGTLTFACVRRRLYHLLRRATLSSHYQVTRPYGHSNSGCSHSTTTLRTSFPVLFAQLAAATAAVVAAISLPSPSLCETAHAGTEERRGERKAMREDGGKGEATSSATCNPSLFEHHDRVDTKLHRAYPEFLKFHLIHETLRGKEKIDGYEVYKDRRDDSIVAYARLGKLLSGHPDIIHGGSIAALLDNTMGVAFFAAKRGNGFTANLTINYKRPITCGTEVKVLARVEKVEGRKVFLRAEIRDAKDEAILYTEAKSLFITSQSPLLKGPKKIDIS

SEQ ID NO: 8 - DNA, 864 bp, Nannochloropsis oculata NIES2145
atgacgcctttggccttcacggtgctcggcaagcttggtggcacgttgacttttgcttgtgtacgacggaggctttatcacttgttacggcgggcaactttgtcctcccattatcaggtcactcggccttacggtcacagcaattccggctgttcacatagcactaccacacttagaaccagcttcccagtcctctttgcgcaattggcagcagccactgctgccgtcgtcgctgccatttccctgccgtcgcctagtctatgcgagacggcccacgccgggactgaggagagacgaggtgagaggaaggcaatgagggaggatggtggaaaaggcgaggccacctcgtctgctacatgcaatccatccttattcgaacatcatgatcgcgtcgacaccaagctgcatcgggcctatcctgaattcctgaagttccaccttatccacgagacgctccgaggcaaagagaaaattgatggctacgaagtttacaaagacaggcgggatgattcaattgtggcgtatgctcgccttggcaaactgctgagcggacaccccgacataatccacggagggtccattgcggctttgctggacaataccatgggagttgcctttttcgccgccaagcgtggcaatggttttacagcaaatctcaccatcaactacaagcgacccatcacgtgtggcaccgaagtcaaagttttagctcgagtagagaaggtggaagggcgcaaggtcttcttgcgggccgagattcgagacgctaaggatgaggctatcctctacactgaagccaaatccctcttcatcacgtctcaaagtcctttattgaagggcccaaagaaaattgatattagctag

SEQ ID NO: 9 - protein, 274 aa, Nannochloropsis gaditana CCMP526
MLCCACKSVHATISVAFIGTRKPHRLPALFPLFLAPARALSHQEPNPATCGTQNSSFSILLKTVVAGSFVGAAFIAGHTAGASCDEVKSPQEVNNVGGGAPVTAPYTVTFASNYHDRVDTKLHRAYPEFLQYHLIHETLRGKEKIEGYEVYKDRRDDSIVAFARLGKLLSGHPDIIHGGSIAALLDNTMGVAFFAANKGNGFTANLTINYKRPIICGTEIKVLARVERFEGRKVFLRAEIRDAKDEAVLYTEATSLFITSQSPLLTGPKKVDIS

SEQ ID NO: 10 - DNA, 825 bp, Nannochloropsis gaditana CCMP526
atgctatgttgcgcctgtaaatcagtgcatgcgactattagtgtcgcctttattggtactcggaagccacatcgtttgcctgcattgtttccattgttccttgccccggcccgagcactcagccatcaggagccgaaccctgcaacgtgcgggacgcaaaactcatccttctcgatcttgttgaaaacggtagtagcaggatcattcgtcggtgcggcattcatcgctgggcatacagcaggggctagctgtgatgaagtaaagtctccgcaggaggtgaacaatgtaggaggcggcgccccagtgactgccccctacacggtcacttttgcgtccaattatcatgatcgagtggacacaaaacttcatagagcttatcctgagtttttacagtaccatcttattcatgaaacgcttcgaggcaaggaaaagatagagggctacgaggtgtacaaagataggcgtgacgattctatcgtagcatttgctcgcctcgggaagcttctcagcgggcatccggatataatccatggaggctctatagccgccttactcgacaacactatgggcgtggcattcttcgctgccaataaaggtaatggcttcactgccaacctcacaatcaattacaagaggccgatcatttgtggcaccgagatcaaggtcttggcccgagtggagcggtttgaaggacgcaaggttttcctacgagcagagattcgagatgctaaggacgaggcagtgttgtacacggaagccacatccctcttcataacttcacaaagtcctctgcttacgggaccgaagaaggtggacatcagttag

SEQ ID NO: 11 - protein, 285 aa, Nannochloropsis granulata NIES2588
MTPLAFTALGEVGGMLAAACVRRKLHHLLRRAASSSQVTRPYSHSTANSTHSTTTLSNSFPVLFAQLAAAAAAVMAATSLSSPSLCETAHTNTEERGGEGEAMREKGGEGEATSSATCAPSFFEHHDRVDTKLHRAYPEFLKFHLIHETLRGKEKIDGYEVYKNRRDDSVVAYARLGKLLSGHPDIIHGGSIAALLDNTMGVAFFAAKRGNGFTANLTINYKRPITCGTEVKVLARVEKVEGRKVFLRAEIRDAKDEAILYTEANSLFITSQSPLLKGPKKIDIS

SEQ ID NO: 12 - DNA, 858 bp, Nannochloropsis granulata NIES2588
atgacgcctttggccttcacggcgctcggcgaggtcggtggcatgttggctgctgcctgtgtacgacggaagcttcatcacttgttgcggcgggcagcttcgtcctcccaggtcactcgaccttacagtcacagcaccgccaacagcacacatagcaccaccacacttagcaacagctttccagtcctctttgcgcaactcgcagcagccgctgctgccgtcatggctgccacttccctgtcgtcgcccagtctatgtgagacggcccacaccaatactgaggagagaggaggcgaaggggaggcaatgagggagaagggtggggaaggcgaggccacttcgtctgctacatgcgctccatctttcttcgagcatcatgatcgcgtcgacacgaagctgcatcgggcctatcccgagtttctgaagttccacctcatccacgagacgctccgagggaaagagaaaattgatggctacgaagtatacaaaaacaggcgggacgattcagttgtggcgtatgctcgcctgggcaaactgctgagcggacaccctgacataattcacggagggtccatcgctgctttgctggacaacaccatgggagttgcctttttcgccgccaagcgcggcaatggtttcacagcaaatctcaccatcaactacaagcgacccatcacgtgtggcaccgaggtcaaagttctggctcgagtagagaaggtggaggggcgcaaggtctttttgcgggctgagatcagggacgccaaggatgaggctatcctttacactgaagccaactccctcttcatcacgtcgcaaagccctctattgaagggcccaaagaaaattgacattagctag

SEQ ID NO: 13 - protein, 233 aa, Symbiodinium microadriaticum
MAFRLCSLSRRFAAHAQQVLRKEAGFEFRASCIAITAGISAGWCMQQAARAEGIWTPHLGEEAKLLNLQREMALRDRHDKQFVWQTCSGQGKIEDCRIYHCKREEVDREVSLDAPEMVEGKTRICAVMRVGDELNGHPGLLHGGFTAAVLDDFTGLATWMEKQAQALDKDAAIFTAHMDLSYRRPLKAKSEYLVEVCVDRVERQKKVFLNAAIYDKDSHACVKAKVLYIVKKK

SEQ ID NO: 14 - DNA, 702 bp, Symbiodinium microadriaticum
atggctttcaggctatgctctctttcccggcggtttgctgcgcacgcgcagcaggtgctgcggaaggaggctggctttgagttccgcgcaagctgcatcgccattaccgctggcatctctgctggatggtgcatgcagcaggcagcgcgggcggagggcatctggactccgcacctgggcgaggaggccaagttgttgaacctccagcgcgagatggcgctgagagacagacacgacaagcaatttgtgtggcagacctgcagtggccagggcaaaattgaggactgccgcatatatcactgcaagcgagaagaagttgatcgtgaggtttcgctggacgcgccggaaatggtggagggcaaaacacggatttgtgcagtgatgcgcgttggcgacgagctgaacggccatcctgggcttttgcatggcggcttcactgccgccgtgctggacgatttcacaggcctggcgacctggatggagaagcaagcgcaggcgctggacaaggatgcggccattttcaccgctcacatggatctcagctatcggcgacccctgaaggcgaagtcggagtacttggttgaggtttgcgttgaccgtgttgagcggcaaaagaaggtctttctgaatgctgccatctatgacaaggacagccatgcctgcgtgaaagcaaaggtgttgtacatcgtcaaaaagaagtga

SEQ ID NO: 15 - DNA, 1747 bp, Brassica rapa
gatatcactacaatgtcggagagacaaggctgcgccagcatatacaaaagggaaatgaagatggccttttgattagctgtgtagcatcagcagctaatctctgggctctcatcatggatgctggaactggattcacttctcaagtttatgagttgtcaccggtcttcctacacaaggtaataatcagttgaagcaattaagaatcaatttgatttgtagtaaactaagaagaacttaccttatgttttccccgcaggactggattatggaacaatgggaaaagaactactatataagctccatagctggttcagataacgggagctctttagttgttatgtcaaaaggttagtgtttagtgaataataaacttataccacaaagtcttcattgacttatttatatacttgttgtgaattgctaggaactacttattctcagcagtcatacaaagtgagtgactcatttccgttcaagtggataaataagaaatggaaagaagattttcatgtaacctccatgacaactgctggtaatcgttggggtgtggtaatgtcgaggaactctggcttctctgatcaggtaggtttttgtctcttatggtctgggggtttttatttcccctgatagtctaatatgataaactctgcgttgtgaaaggtggtggagcttgactttttgtacccaagcgatgggatacataggaggtgggagaatgggtatagaataacatcaatggcagcaactgcggatcaagcagctttcatattaagcataccaaagcgtaagatggtggatgaaactcaagagactctccgcaccaccgcctttccaagtactcatgtcaaggttggtttctttagctttgaacacagatttggatctttttgttttgtttccatatacttaggacctgagagcttttggttgatttttttttcaggacaaatgggcgaagaatctgtacattgcatcaatatgctatggcaggacagtgtgctgatacacacttaagcatcatgtggaaagccaaagacaattggagcgagactcagggtcgtcataataccaatcaaagacgtaaaaccagacgcaacctctttggttgaatgtaatgaaagggatgtgtcttggtatgtatgtacgaataacaaaagagaagatggaattagtagtagaaatatttgggagctttttaagcccttcaagtgtgctttttatcttattgatatcatccatttgcgttgtttaatgcgtctctagatatgttcctatatctttctcagtgtctgataagtgaaatgtgagaaaaccataccaaaccaaaatattcaaatcttatttttaataatgttgaatcactcggagttgccaccttctgtgccaattgtgctgaatctatcacactagaaaaaaacatttcttcaaggtaatgacttgtggactatgttctgaattctcattaagtttttattttctgaagtttaagtttttaccttctgttttgaaatatatcgttcataagatgtcacgccaggacatgagctacacatcgcacatagcatgcagatcaggacgatttgtcactcacttcaaacacctaagagcttctctctcacagcgcacacacatatgcatgcaatatttacacgtgatcgccatgcaaatctccattctcacctataaattagagcctcggcttcactctttactcaaaccaaaactcatcactacagaacatacacaa

SEQ ID NO: 16 - DNA, 1255 bp, Brassica rapa
gtgtgtataccacggtgatatgagtgtggttgttgatgtatgttaacactacatagtcatggtgtgtgttccataaataatgtactaatgtaataagaactactccgtagacggtaataaaagagaagtttttttttttactcttgctactttcctataaagtgatgattaacaacagatacaccaaaaagaaaacaattaatctatattcacaatgaagcagtactagtctattgaacatgtcagattttctttttctaaatgtctaattaagccttcaaggctagtgatgataaaagatcatccaatgggatccaacaaagactcaaatctggttttgatcagatacttcaaaactatttttgtattcattaaattatgcaagtgttcttttatttggtgaagactctttagaagcaaagaacgacaagcagtaataaaaaaaacaaagttcagttttaagatttgttattgacttattgtcatttgaaaaatatagtatgatattaatatagttttatttatataatgcttgtctattcaagatttgagaacattaatatgatactgtccacatatccaatatattaagtttcatttctgttcaaacatatgatagatggtcaaatgattatgagttttgttatttacctgaagaaagataagtgagcttcgagtttctgaagggtacgtgatcttcatttcttggctaaaagcgaatatgacatcacctagagaaagccgataatagtaaactctgttcttggtttttggtttaatcaaaccgaaccggtagctgagtgtcaagtcagcaaacatcgcaaaccatatgtcaattcgttagattcccggtttaagttgtaaaccggtatttcatttggtgaaaaccctagaagccagccaccctttttaatctaatttttgtaaacgagaagtcaccacacctctccactaaaaccctgaaccttactgagagaagcagagcgcagctcaaagaacaaataaaacccgaagatgagaccaccacgtggcggcgggagcttcaggggacggggaggaagagatggcggcggacgctttggtggcggcggcggacgttttggtggcggcggtggacgctttggtggcggcggtggacgctttggtggtggtggatatcgtgacgaagggcctcccagcgaagtcattggttcgtttactctttacttagtcgaatcttattcttgctctgctcgttgttttaccgataaagctt

SEQ ID NO: 17 - DNA, 240 bp, Umbellularia californica
atggccaccacctctttagcttccgctttctgctcgatgaaagctgtaatgttggctcgtgatggccggggcatgaaacccaggagcagtgatttgcagctgagggcgggaaatgcgccaacctctttgaagatgatcaatgggaccaagttcagttacacggagagcttgaaaaggttgcctgactggagcatgctctttgcagtgatcacaaccatcttttcggctgctgagaagcag

SEQ ID NO: 18 - DNA, 836 bp, Streptomyces hygroscopicus
atgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgatgacccgggtaccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgg

SEQ ID NO: 19 - DNA, 1720 bp, Brassica napus
taccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctttcttcatcggtgattgattcctttaaagacttatgtttcttatcttgcttctgaggcaagtattcagttaccagttaccacttatattctggactttctgactgcatcctcatttttccaacattttaaatttcactattggctgaatgcttcttctttgaggaagaaacaattcagatggcagaaatgtatcaaccaatgcatatatacaaatgtacctcttgttctcaaaacatctatcggatggttccatttgctttgtcatccaattagtgactactttatattattcactcctctttattactattttcatgcgaggttgccatgtacattatatttgtaaggattgacgctattgagcgtttttcttcaattttctttattttagacatgggtatgaaatgtgtgttagagttgggttgaatgagatatacgttcaagtgaatggcataccgttctcgagtaaggatgacctacccattcttgagacaaatgttacattttagtatcagagtaaaatgtgtacctataactcaaattcgattgacatgtatccattcaacataaaattaaaccagcctgcacctgcatccacatttcaagtattttcaaaccgttcggctcctatccaccgggtgtaacaagacggattccgaatttggaagattttgactcaaattcccaatttatattgaccgtgactaaatcaactttaacttctataattctgattaagctcccaatttatattcccaacggcactacctccaaaatttatagactctcatccccttttaaaccaacttagtaaacgtttttttttttaattttatgaagttaagtttttaccttgtttttaaaaagaatcgttcataagatgccatgccagaacattagctacacgttacacatagcatgcagccgcggagaattgtttttcttcgccacttgtcactcccttcaaacacctaagagcttctctctcacagcacacacatacaatcacatgcgtgcatgcattattacacgtgatcgccatgcaaatctcctttatagcctataaattaactcatccgcttcactctttactcaaaccaaaactcatcaatacaaacaagattaaaaacatacacgaggatcc

SEQ ID NO: 20 - DNA, 30 bp, Artificial Sequence
  PCR primer No.1
gatatcactacaatgtcggagagacaaggc

SEQ ID NO: 21 - DNA, 30 bp, Artificial Sequence
  PCR primer No.2
ttgtgtatgttctgtagtgatgagttttgg

SEQ ID NO: 22 - DNA, 28 bp, Artificial Sequence
  PCR primer No.3
agtgtgtataccacggtgatatgagtgt

SEQ ID NO: 23 - DNA, 29 bp, Artificial Sequence
  PCR primer No.4
aagctttatcggtaaaacaacgagcagag

SEQ ID NO: 24 - DNA, 47 bp, Artificial Sequence
  PCR primer No.5
gggggtcgacgatatcactacaatgtcggagagacaaggctgcgcca

SEQ ID NO: 25 - DNA, 57 bp, Artificial Sequence
  PCR primer No.6
gctaaagaggtggtggccatttgtgtatgttctgtagtgatgagttttggtttgagt

SEQ ID NO: 26 - DNA, 45 bp, Artificial Sequence
  PCR primer No.7
ccccccgggaagctttatcggtaaaacaacgagcagagcaagaat

SEQ ID NO: 27 - DNA, 47 bp, Artificial Sequence; identical to SEQ ID NO: 24
  PCR primer No.8

SEQ ID NO: 28 - DNA, 50 bp, Artificial Sequence
  PCR primer No.9
catatgccgcggccgcccactagtttgtgtatgttctgtagtgatgagtt

SEQ ID NO: 29 - DNA, 49 bp, Artificial Sequence
  PCR primer No.10
actagtgggcggccgcggcatatggtgtgtataccacggtgatatgagt

SEQ ID NO: 30 - DNA, 45 bp, Artificial Sequence; identical to SEQ ID NO: 26
  PCR primer No.11

SEQ ID NO: 31 - DNA, 30 bp, Artificial Sequence
  PCR primer No.12
gcggccgcatggccaccacctctttagctt

SEQ ID NO: 32 - DNA, 39 bp, Artificial Sequence
  PCR primer No.13
gcggccgctctagattggtccactgcttctcagcagccg

SEQ ID NO: 33 - DNA, 37 bp, Artificial Sequence
  PCR primer No.14
tggaccaatctagagctcgattccaagaagagggggg

SEQ ID NO: 34 - DNA, 37 bp, Artificial Sequence
  PCR primer No.15
atatgccgcggccgctcatttactctcagttgggtgc

SEQ ID NO: 35 - DNA, 25 bp, Artificial Sequence
  PCR primer No.16
ctctagattggtccactgcttctca

SEQ ID NO: 36 - DNA, 23 bp, Artificial Sequence
  PCR primer No.17
gcggccgcggcatatggtgtgta

SEQ ID NO: 37 - DNA, 33 bp, Artificial Sequence
  PCR primer No.18
atatatatatactagtatgagcccagaacgacg

SEQ ID NO: 38 - DNA, 37 bp, Artificial Sequence
  PCR primer No.19
atatatatatcatatgatcagatctcggtgacgggca

SEQ ID NO: 39 - DNA, 36 bp, Artificial Sequence
  PCR primer No.20
tgacgagttccatatggcgggactctggggttcgaa

SEQ ID NO: 40 - DNA, 37 bp, Artificial Sequence
  PCR primer No.21
catcttgttcactagtgcgaaacgatccagatccggt

SEQ ID NO: 41 - DNA, 32 bp, Artificial Sequence
  PCR primer No.22
taccgaggggaatttatggaacgtcagtggag

SEQ ID NO: 42 - DNA, 31 bp, Artificial Sequence
  PCR primer No.23
actagtggatcctcgtgtatgtttttaatct

SEQ ID NO: 43 - DNA, 31 bp, Artificial Sequence
  PCR primer No.24
actctgagattaacctatggctccccttaaa

SEQ ID NO: 44 - DNA, 31 bp, Artificial Sequence
  PCR primer No.25
gaattcgtaatcatggtcatagctgtttcct

SEQ ID NO: 45 - DNA, 42 bp, Artificial Sequence
  PCR primer No.26
cgaggatccactagtatggccaccacctctttagcttccgct

SEQ ID NO: 46 - DNA, 40 bp, Artificial Sequence
  PCR primer No.27
catgattacgaattcaagctttatcggtaaaacaacgagc

SEQ ID NO: 47 - DNA, 30 bp, Artificial Sequence
  PCR primer No.28
actagtggatcctcgtgtatgtttttaatc

SEQ ID NO: 48 - DNA, 30 bp, Artificial Sequence
  PCR primer No.29
ggcatatggtgtgtataccacggtgatatg

SEQ ID NO: 49 - DNA, 41 bp, Artificial Sequence
  PCR primer No.30
cgaggatccactagtatggccacaagtgctagtatcggggt

SEQ ID NO: 50 - DNA, 44 bp, Artificial Sequence
  PCR primer No.31
tacacaccatatgccttaaggcatgaagggagcaaaaacaacca

SEQ ID NO: 51 - DNA, 37 bp, Artificial Sequence
  PCR primer No.32
cgaggatccactagtatggccgggtactcggtggcgg

SEQ ID NO: 52 - DNA, 41 bp, Artificial Sequence
  PCR primer No.33
tacacaccatatgccctatttgtaaggcgcaaacaagatag

SEQ ID NO: 53 - DNA, 41 bp, Artificial Sequence
  PCR primer No.34
cgaggatccactagtatggacggcctccgccttccctccat

SEQ ID NO: 54 - DNA, 39 bp, Artificial Sequence
  PCR primer No.35
tacacaccatatgcctcatggcttagaaggtgcaaatac

(19) United States
(12) Patent Application Publication
(10) Pub. No.: US 2017/0107545 A1
(43) Pub. Date: Apr. 20, 2017

(54) Method of Producing Medium Chain Fatty Acid Using Beta-Ketoacyl-ACP Synthase

(71) Applicant: Kao Corporation, Chuo-ku, Tokyo (JP)

(72) Inventors: Takuto TOJO, Wakayama-shi, Wakayama (JP); Shinji SUGIHARA, Wakayama-shi, Wakayama (JP)

(73) Assignee: Kao Corporation, Chuo-ku, Tokyo (JP)

(21) Appl. No.: 15/317,347

(22) PCT Filed: Jul. 30, 2015
(86) PCT No.: PCT/JP2015/071666

§ 371 Date: Dec. 8, 2016

(30) Foreign Application Priority Data
Aug. 4, 2014 (JP) 2014-159011

(51) Int. Cl. C12P 7/64; C12N 15/52; C12N 9/16; C12N 9/10

(52) U.S. Cl. CPC C12P 7/6418; C12N 9/1029; C12Y 203/01041; C12N 15/52; C12Y 301/02014; C12N 9/16

(57) ABSTRACT

[Problems] To provide a method of producing a medium chain fatty acid or a lipid containing this fatty acid as a component by using a β-ketoacyl-ACP synthase, and a gene, a protein and a transformant for use in this method.

[Means to solve] A method of producing a medium chain fatty acid or a lipid containing this fatty acid as a component, containing the steps of:
introducing a gene encoding the following protein (A) or (B) into a host, and thereby obtaining a transformant, and
collecting a medium chain fatty acid or a lipid containing this fatty acid as a component from the resulting transformant;
and a gene, a protein and a transformant for use in this method:
   (A) A protein consisting of the amino acid sequence set forth in SEQ ID NO: 1; and
   (B) A protein consisting of an amino acid sequence having 90% or more identity with the amino acid sequence set forth in SEQ ID NO: 1, and having medium chain acyl-ACP-specific β-ketoacyl-ACP synthase activity.

### TECHNICAL FIELD

The present invention relates to a β-ketoacyl-ACP synthase, and a method of producing a medium chain fatty acid or a lipid containing this fatty acid as a component using the same.

### BACKGROUND ART

Fatty acids are one of the principal components of lipids. In vivo, fatty acids are bonded to glycerin via an ester bond to form lipids such as triacylglycerol. Further, many animals and plants store and utilize fatty acids as an energy source. These fatty acids and lipids stored in animals and plants are widely utilized for food or industrial use.

For example, higher alcohol derivatives that are obtained by reducing higher fatty acids having approximately 12 to 18 carbon atoms are used as surfactants. Alkyl sulfuric acid ester salts and alkylbenzenesulfonic acid salts are utilized as anionic surfactants. Further, polyoxyalkylene alkyl ethers, alkyl polyglycosides and the like are utilized as nonionic surfactants. These surfactants are used for detergents or disinfectants. Other higher alcohol derivatives, alkylamine salts and mono- or dialkyl-quaternary amine salts are commonly used for fiber treatment agents, hair conditioning agents or disinfectants. Further, benzalkonium type quaternary ammonium salts are commonly used for disinfectants or antiseptics. Moreover, vegetable fats and oils are used also as raw materials of biodiesel fuels.

A fatty acid synthesis pathway of plants is localized in a chloroplast. In the chloroplast, an elongation reaction of the carbon chain is repeated starting from an acetyl-ACP (acyl-carrier-protein), and finally an acyl-ACP (a composite consisting of an acyl group being a fatty acid residue and an ACP) having 16 or 18 carbon atoms is synthesized. A β-ketoacyl-ACP synthase (β-ketoacyl-acyl-carrier-protein synthase: hereinafter, also referred to as “KAS”) is an enzyme involved in control of chain length of the acyl group, among enzymes involved in the fatty acid synthesis pathway. In the plants, four kinds of KASs having different function respectively, namely KAS I, KAS II, KAS III and KAS IV are known to exist. Among these, KAS III functions in a stage of starting a chain length elongation reaction to elongate the acetyl-ACP (or acetyl-CoA) having 2 carbon atoms to the β-ketoacyl-ACP having 4 carbon atoms. In the subsequent elongation reaction, KAS I, KAS II and KAS IV are involved. KAS I is mainly involved in the elongation reaction to the palmitoyl-ACP having 16 carbon atoms, and KAS II is mainly involved in the elongation reaction to the stearoyl-ACP having 18 carbon atoms. On the other hand, it is believed that KAS IV is involved in the elongation reaction to medium chain acyl-ACP having 6 to 14 carbon atoms.

Currently, information on KSV IV of plants is hardly obtained, and only a limited example of report on Cuphea in dicotyledonous plants is found (see Patent Literature 1 and Non-Patent Literature 1).

### CITATION LIST

### Patent Literatures

Patent Literature 1: WO 98/46776

### Non-Patent Literatures

Non-Patent Literature 1: Dehesh K, et al., The Plant Journal., 1998, vol. 15(3), p. 383-390

### SUMMARY OF INVENTION

The present invention relates to a method of producing a medium chain fatty acid or a lipid containing this fatty acid as a component, containing the steps of: introducing a gene encoding the following protein (A) or (B) into a host, and thereby obtaining a transformant, and collecting a medium chain fatty acid or a lipid containing this fatty acid as a component from the resulting transformant (hereinafter, the method is referred to as “the producing method of the present invention”).

(A) A protein consisting of the amino acid sequence set forth in SEQ ID NO: 1.
(B) A protein consisting of an amino acid sequence having 90% or more identity with the amino acid sequence set forth in SEQ ID NO: 1, and having a medium chain acyl-ACP-specific β-ketoacyl-ACP synthase activity.

The present invention also relates to the protein (A) or (B) (hereinafter, referred to as “the β-ketoacyl-ACP synthase of the present invention”) and a gene encoding the protein (hereinafter, referred to as “the β-ketoacyl-ACP synthase gene of the present invention”).

The present invention also relates to a transformant obtained by introducing a gene encoding the protein (A) or (B) into a host (hereinafter, referred to as “the transformant of the present invention”).

Other and further features and advantages of the invention will appear more fully from the following description.

### MODE FOR CARRYING OUT THE INVENTION

The present invention is contemplated for providing a method of producing a medium chain fatty acid or a lipid containing this fatty acid as a component by using a β-ketoacyl-ACP synthase derived from a plant. Furthermore, the present invention is contemplated for providing a novel β-ketoacyl-ACP synthase derived from a plant.

The present inventors made extensive studies about the β-ketoacyl-ACP synthases of a plant. As a result, they found novel fβ-ketoacyl-ACP synthases derived from Cocos nucifera. Then, the present inventors found that when a host was transformed by using them, productivity of medium chain fatty acids or esters thereof is significantly improved in the transformant. The present invention was completed based on these findings.

The transformant of the present invention is excellent in ability to produce the medium chain fatty acids and lipids containing as components the medium chain fatty acids. The production method of the present invention using the transformant can produce medium chain fatty acids, and lipids containing as components the medium chain fatty acids. Further, the β-ketoacyl-ACP synthase and the gene encoding the same of the present invention can be used for synthesizing a medium chain acyl-ACP.

The β-ketoacyl-ACP synthase, the gene encoding this β-ketoacyl-ACP synthase, the transformant and the production method of the present invention can be suitably used for the industrial production of medium chain fatty acids and lipids containing as components the medium chain fatty acids.

In the present specification, the term “lipid(s)” covers simple lipids, complex lipids and derived lipids. Specifically, “lipid(s)” covers fatty acids, aliphatic alcohols, hydrocarbons (such as alkanes), neutral lipids (such as triacylglycerol), wax, ceramides, phospholipids, glycolipids, sulfolipids and the like.

In the present specification, the description of “Cx:y” for the fatty acid or the acyl group constituting the fatty acid means that the number of carbon atoms is “x” and the number of double bonds is “y”. The description of “Cx” means a fatty acid or an acyl group having “x” as the number of carbon atoms.

Hereinafter, the β-ketoacyl-ACP synthase, and the transformant and the method of producing a lipid using the same are described below in order.

### 1. β-Ketoacyl-ACP Synthase

The fβ-ketoacyl-ACP synthase of the present invention includes a protein consisting of the amino acid sequence set forth in SEQ ID NO: 1, and a protein functionally equivalent to the protein. Specifically, the β-ketoacyl-ACP synthase of the present invention includes the following protein (A) or (B) is used.

(A) A protein consisting of the amino acid sequence set forth in SEQ ID NO: 1.
(B) A protein consisting of an amino acid sequence having 90% or more identity with the amino acid sequence set forth in SEQ ID NO: 1, and having medium chain acyl-ACP-specific β-ketoacyl-ACP synthase activity.

The protein consisting of the amino acid sequence set forth in SEQ ID NO: 1 is a novel β-ketoacyl-ACP synthase derived from Cocos nucifera of monocotyledon.

The β-ketoacyl-ACP synthase is an enzyme involved in control of chain length of an acyl group in the fatty acid synthesis pathway. The fatty acid synthesis pathway of plants is localized in the chloroplast. In the chloroplast, the elongation reaction of the carbon chain is repeated starting from the acetyl-ACP (or acetyl-CoA), and finally an acyl-ACP having 16 or 18 carbon atoms is synthesized. Then, an acyl-ACP thioesterase (hereinafter, also simply referred to as “TE”) hydrolyzes the thioester bond of the acyl-ACP to form free fatty acids.

In the first stage of the fatty acid synthesis, an acetoacetyl-ACP is formed by a condensation reaction between the acetyl-ACP (or acetyl-CoA) and a malonyl-ACP. The β-ketoacyl-ACP synthase catalyzes the reaction. Then, the keto group of the acetoacetyl-ACP is reduced by a β-ketoacyl-ACP reductase, to produce a hydroxybutyryl-ACP. Subsequently, the hydroxybutyryl-ACP is dehydrated by a β-hydroxyacyl-ACP dehydrase, to produce a crotonyl-ACP. Finally, the crotonyl-ACP is reduced by an enoyl-ACP reductase, to produce a butyryl-ACP. The butyryl-ACP in which two carbon atoms are added to the carbon chain of the acyl group of the acetyl-ACP by a series of reactions. Hereinafter, the similar reactions are repeated to cause elongation of the carbon chain of the acyl-ACP, and an acyl-ACP having 16 or 18 carbon atoms is finally synthesized.

The protein (A) or (B) has β-ketoacyl-ACP synthase activity. In the present invention, an expression “β-ketoacyl-ACP synthase activity” means the activity to catalyze the condensation reaction of the acetyl-ACP or the acyl-ACP with the malonyl-ACP.

The β-ketoacyl-ACP synthase activity of a protein can be measured by, for example, introducing a DNA produced by linking a gene encoding the protein to the downstream of a promoter which functions in a host cell such as Escherichia coli, into a host cell which lacks a fatty acid degradation system, culturing the thus-obtained cell under the conditions suitable for the expression of the introduced gene, and analyzing any change caused thereby in the fatty acid composition of the cell or the cultured liquid by using a gas chromatographic analysis or the like. Alternatively, the β-ketoacyl-ACP synthase activity can be measured by introducing a DNA produced by linking a gene encoding the protein to the downstream of a promoter which functions in a host cell such as Escherichia coli, into a host cell, culturing the thus-obtained cell under the conditions suitable for the expression of the introduced gene, and subjecting a disruption liquid of the cell to a chain length elongation reaction which uses acyl-ACPs, as substrates, prepared according to the method described in the above-mentioned Non-Patent Literature 1 (Dehesh et al., The Plant Journal., 1998, vol. 15(3), p. 383-390).

The β-ketoacyl-ACP synthase (hereinafter, also simply referred to as “KAS”) catalyzes the condensation reaction of the acyl-ACP with the malonyl-ACP, and is categorized into KAS I, KAS II, KAS III and KAS IV according to substrate specificity. KAS III uses an acetyl-ACP (or acetyl-CoA) having 2 carbon atoms as the substrate to catalyze the elongation reaction that the acetyl-ACP having 2 carbon atoms is converted to the acyl-ACP having 4 carbon atoms. KAS I mainly catalyzes the elongation reaction that the acyl-ACP having 4 carbon atoms is converted to the acyl-ACP having 16 carbon atoms, to synthesize the palmitoyl-ACP having 16 carbon atoms. KAS II mainly catalyzes the elongation reaction that the acyl-ACP having 16 carbon atoms is converted to the acyl-ACP having 18 carbon atoms, to synthesize the stearoyl-ACP having 18 carbon atoms. KAS IV catalyzes the elongation reaction that the acyl-ACP having 6 carbon atoms is converted to the acyl-ACP having 14 carbon atoms, to synthesize a medium chain acyl-ACP.

As shown in Examples mentioned later, the β-ketoacyl-ACP synthase specified in the protein (A) selectively synthesizes the medium chain acyl-ACP, and is considered to be KAS IV.

In the present invention, the term “medium chain acyl-ACP-specific β-ketoacyl-ACP synthase” means a β-ketoacyl-ACP synthase which mainly uses an acyl-ACP having 4 to 12 carbon atoms as the substrate to selectively catalyze the elongation reaction for the synthesis of the medium chain acyl-ACP having 6 to 14 carbon atoms. Hereinafter, the medium chain acyl-ACP-specific β-ketoacyl-ACP synthase is also referred to as a medium chain-specific β-ketoacyl-ACP synthase.

Moreover, in the present invention, the term “medium chain” means that the number of carbon atoms of the acyl group is 6 or more and 14 or less.

The specificity of the β-ketoacyl-ACP synthase to the medium chain acyl-ACP can be confirmed by, for example, introducing a DNA produced by linking a gene encoding the protein to the downstream of a promoter which functions in a host cell such as Escherichia coli, into a host cell which lacks a fatty acid degradation system, culturing the thus-obtained cell under the conditions suitable for the expression of the introduced gene, analyzing any change caused thereby in the fatty acid composition of the cell or the cultured liquid by using a method such as a gas chromatographic analysis, and confirming the increase of the medium chain fatty acids. Alternatively, the specificity to the medium chain acyl-ACP can be confirmed by allowing, in the abode-described system, coexpression of medium chain-specific acyl-ACP thioesterase described later, and confirming the increase of the medium chain fatty acids in comparison with the specificity during medium chain-specific acyl-ACP thioesterase single expression. Alternatively, the specificity to the medium chain acyl-ACP can be confirmed by introducing a DNA produced by linking a gene encoding the protein to the downstream of a promoter which functions in a host cell such as Escherichia coli, into a host cell, culturing the thus-obtained cell under the conditions suitable for the expression of the introduced gene, and subjecting a disruption liquid of the cell to a chain length elongation reaction which uses acyl-ACPs, as substrates, prepared according to the method described in the above-mentioned Non-Patent Literature 1 (Dehesh et al., The Plant Journal., 1998, vol. 15(3), p. 383-390).

In the protein (B), the identity with the amino acid sequence set forth in SEQ ID NO: 1 is preferably 95% or more, more preferably 96% or more, further preferably 97% or more, further more preferably 98% or more, and further more preferably 99% or more, in view of medium chain specificity.

In the present specification, the identity of the amino acid sequence and nucleotide sequence is calculated through the Lipman-Pearson method (see Science, 227, pp. 1435, (1985)). Specifically, the identity can be determined through use of a homology analysis (homology search) program of genetic information processing software Genetyx-Win (Software Development Co., Ltd.) with Unit size to compare (ktup) being set to 2.

As the amino acid sequence of the protein (B), an amino acid sequence in which mutation is introduced in the amino acid sequence set forth in SEQ ID NO: 1, that is, an amino acid sequence in which 1 or several amino acids are deleted, substituted, inserted or added in the amino acid sequence set forth in SEQ ID NO: 1, is also preferable. From the point of view of the medium chain specificity, the amino acid sequence of the protein (B) is particularly preferably an amino acid sequence in which preferably 1 to 10 amino acids, more preferably 1 to 5 amino acids, more preferably 1 to 3 amino acids, more preferably 1 or 2 amino acids, and further preferably 1 amino acid, are deleted, substituted, inserted or added in the amino acid sequence set forth in SEQ ID NO: 1.

A method of introducing the mutation such as deletion, substitution, addition or insertion into an amino acid sequence includes a method of, for example, introducing a mutation into a nucleotide sequence encoding the amino acid sequence. The method of introducing a mutation into a nucleotide sequence is described later.

There are no particular limitations on the method for obtaining the above-described protein, and the protein may be obtained by chemical techniques or genetic engineering techniques that are ordinarily carried out. For example, a natural product-derived protein can be obtained through isolation, purification and the like from Cocos nucifera. Furthermore, protein synthesis may be carried out by chemical synthesis, or a recombinant protein may also be produced by gene recombination technologies. In the case of producing a recombinant protein, the β-ketoacyl-ACP synthase gene described below can be used.

### 2. β-Ketoacyl-ACP Synthase Gene

The β-ketoacyl-ACP synthase gene of the present invention is a gene encoding the protein (A) or (B).

Examples of the gene encoding the amino acid sequence set forth in SEQ ID NO: 1 include a nucleotide sequence set forth in SEQ ID NO: 2. The nucleotide sequence set forth in SEQ ID NO: 2 is an example of a nucleotide sequence of a gene encoding wild-type β-ketoacyl-ACP synthase derived from Cocos nucifera.

Specific examples of the gene encoding the protein (A) or (B) include a gene consisting of the following DNA (a) or (b), but the present invention is not limited thereto.

(a) A DNA consisting of the nucleotide sequence set forth in SEQ ID NO: 2.
(b) A DNA consisting of a nucleotide sequence having 90% or more identity with the nucleotide sequence set forth in SEQ ID NO: 2, and encoding a protein having a medium chain acyl-ACP-specific β-ketoacyl-ACP synthase activity.

In the DNA (b), from the point of view of the medium chain specificity, the identity with the nucleotide sequence set forth in SEQ ID NO: 2 is preferably 95% or more, more preferably 96% or more, more preferably 97% or more, more preferably 98% or more, and more preferably 99% or more.

As the nucleotide sequence of the DNA (b), a nucleotide sequence in which mutation is introduced in the nucleotide sequence set forth in SEQ ID NO: 2, that is, a nucleotide sequence in which 1 or several nucleotides are deleted, substituted, inserted or added in the nucleotide sequence set forth in SEQ ID NO: 2, is also preferable. From the point of view of the medium chain specificity, the nucleotide sequence of the DNA (b) is particularly preferably a nucleotide sequence in which preferably 1 to 10 nucleotides, more preferably 1 to 5 nucleotides, more preferably 1 to 3 nucleotides, more preferably 1 or 2 nucleotides, and further preferably 1 nucleotide, are deleted, substituted, inserted or added in the nucleotide sequence set forth in SEQ ID NO: 2.

A method of introducing the mutation such as deletion, substitution, addition or insertion into a nucleotide sequence includes a method of, for example, introducing a site-specific mutation. Specific examples of the method of introducing the site-specific mutation include a method of utilizing the Splicing overlap extension (SOE) PCR (Horton et al., Gene 77, 61-68, 1989), the ODA method (Hashimoto-Gotoh et al., Gene, 152, 271-276, 1995), and the Kunkel method (Kunkel, T. A., Proc. Natl. Acad. Sci. USA, 1985, 82, 488). Further, commercially available kits such as Site-Directed Mutagenesis System Mutan-SuperExpress Km kit (manufactured by Takara Bio), Transformer TM Site-Directed Mutagenesis kit (manufactured by Clonetech Laboratories), and KOD-Plus-Mutagenesis kit (manufactured by Toyobo) can also be utilized. Furthermore, a gene containing a desired mutation can also be obtained by introducing a genetic mutation at random, and then performing an evaluation of the enzyme activities and a gene analysis thereof by an appropriate method.

A method of obtaining the β-ketoacyl-ACP synthase gene is not particularly limited, and the β-ketoacyl-ACP synthase gene can be obtained by ordinary genetic engineering techniques. For example, the β-ketoacyl-ACP synthase gene can be obtained by artificial synthesis based on the amino acid sequence set forth in SEQ ID NO: 1 or the nucleotide sequence set forth in SEQ ID NO: 2. The artificial synthesis of a gene can be achieved by utilizing, for example, the services of Invitrogen or the like. Furthermore, the gene can also be obtained by cloning from Cocos nucifera. The cloning can be carried out by, for example, the methods described in Molecular Cloning—A LABORATORY MANUAL THIRD EDITION [Joseph Sambrook, David W. Russell, Cold Spring Harbor Laboratory Press (2001)] or the like.

### 3. Acyl-ACP Thioesterase

The transformant of the present invention preferably has a gene encoding an acyl-ACP thioesterase, in addition to the gene encoding the protein (A) or (B), introduced into a host.

The acyl-ACP thioesterase is an enzyme that hydrolyzes the thioester bond of the acyl-ACP synthesized by a fatty acid synthetase such as the β-ketoacyl-ACP synthase to produce free fatty acids. The function of the acyl-ACP thioesterase terminates the fatty acid synthesis on the ACP, and then the thus-hydrolyzed fatty acids are supplied to the synthesis of triglyceride and the like.

Therefore, lipid productivity of the transformant, particularly, productivity of fatty acids can be further improved by introducing the β-ketoacyl-ACP synthase gene and the acyl-ACP thioesterase gene into the host.

The acyl-ACP thioesterase that can be used in the present invention only needs to be the protein having acyl-ACP thioesterase activity. In the present invention, the “acyl-ACP thioesterase activity” means an activity of hydrolyzing the thioester bond of the acyl-ACP.

To date, several acyl-ACP thioesterases having different reaction specificities depending on the number of carbon atoms and the number of unsaturated bonds of the acyl group (fatty acid residue) constituting the acyl-ACP substrate are identified. Therefore, they are considered to be an important factor in determining the fatty acid composition of an organism, in a manner similar to the β-ketoacyl-ACP synthase.

The acyl-ACP thioesterase is preferably a thioesterase having the specificity to the medium chain acyl-ACP (hereinafter, also referred to as “medium chain-specific acyl-ACP thioesterase”). In the present specification, the “medium chain acyl-ACP-specific” acyl-ACP thioesterase means an acyl-ACP thioesterase having an activity of selectively hydrolyzing the thioester bond of the acyl-ACP having 6 to 14 carbon atoms.

The productivity of medium chain fatty acids can be further improved by using the medium chain-specific acyl-ACP thioesterase. In particular, when a host originally having no medium chain-specific acyl-ACP thioesterase is used, introduction of the medium chain-specific acyl-ACP thioesterase is effective.

In the present invention, any known acyl-ACP thioesterases and proteins functionally equivalent to the known acyl-ACP thioesterases can be used. The acyl-ACP thioesterase to be used can be appropriately selected according to a kind of host or the like.

Specific examples thereof include an acyl-ACP thioesterase derived from Umbellularia californica (GenBank AAA34215.1); an acyl-ACP thioesterase derived from Cuphea calophylla subsp. mesostemon (GenBank ABB71581); an acyl-ACP thioesterase derived from Cocos nucifera (CnFatB3: see Jing et al. BMC Biochemistry 2011, 12:44, SEQ ID NO: 5, the nucleotide sequence of the gene encoding this thioesterase: SEQ ID NO: 6); an acyl-ACP thioesterase derived from Cinnamomum camphora (GenBank AAC49151.1); an acyl-ACP thioesterase derived from Myristica fragrans (GenBank AAB71729); an acyl-ACP thioesterase derived from Myristica fragrans (GenBank AAB71730); an acyl-ACP thioesterase derived from Cuphea lanceolate (GenBank CAA54060); an acyl-ACP thioesterase derived from Cuphea hookeriana (GenBank Q39513); an acyl-ACP thioesterase derived from Ulumus americana (GenBank AAB71731); an acyl-ACP thioesterase derived from Sorghum bicolor (GenBank EER87824); an acyl-ACP thioesterase derived from Sorghum bicolor (GenBank EER88593); an acyl-ACP thioesterase derived from Cocos nucifera (CnFatB1: see Jing et al. BMC Biochemistry 2011, 12:44); an acyl-ACP thioesterase derived from Cocos nucifera (CnFatB2: see Jing et al. BMC Biochemistry 2011, 12:44); an acyl-ACP thioesterase derived from Cuphea viscosissima (CvFatB1: see Jing et al. BMC Biochemistry 2011, 12:44); an acyl-ACP thioesterase derived from Cuphea viscosissima (CvFatB2: see Jing et al. BMC Biochemistry 2011, 12:44); an acyl-ACP thioesterase derived from Cuphea viscosissima (CvFatB3: see Jing et al. BMC Biochemistry 2011, 12:44); an acyl-ACP thioesterase derived from Elaeis guineensis (GenBank AAD42220); an acyl-ACP thioesterase derived from Desulfovibrio vulgaris (GenBank ACL08376); an acyl-ACP thioesterase derived from Bacteroides fragilis (GenBank CAH09236); an acyl-ACP thioesterase derived from Parabacteriodes distasonis (GenBank ABR43801); an acyl-ACP thioesterase derived from Bacteroides thetaiotaomicron (GenBank AA077182); an acyl-ACP thioesterase derived from Clostridium asparagiforme (GenBank EEG55387); an acyl-ACP thioesterase derived from Bryanthella formatexigens (GenBank EET61113); an acyl-ACP thioesterase derived from Geobacillus sp. (GenBank EDV77528); an acyl-ACP thioesterase derived from Streptococcus dysgalactiae (GenBank BAH81730); an acyl-ACP thioesterase derived from Lactobacillus brevis (GenBank ABJ63754); an acyl-ACP thioesterase derived from Lactobacillus plantarum (GenBank CAD63310); an acyl-ACP thioesterase derived from Anaerococcus tetradius (GenBank EE182564); an acyl-ACP thioesterase derived from Bdellovibrio bacteriovorus (GenBank CAE80300); an acyl-ACP thioesterase derived from Clostridium thermocellum (GenBank ABN54268); an acyl-ACP thioesterase derived from Nannochloropsis oculata (SEQ ID NO: 7, the nucleotide sequence of the gene encoding this thioesterase:SEQ ID NO: 8); an acyl-ACP thioesterase derived from Nannochloropsis gaditana (SEQ ID NO: 9, the nucleotide sequence of the gene encoding this thioesterase:SEQ ID NO: 10); an acyl-ACP thioesterase derived from Nannochloropsis granulata (SEQ ID NO: 11, the nucleotide sequence of the gene encoding this thioesterase:SEQ ID NO: 12); and an acyl-ACP thioesterase derived from Symbiodinium microadriaticum (SEQ ID NO: 13, the nucleotide sequence of the gene encoding this thioesterase: SEQ ID NO: 14).

Moreover, as the proteins functionally equivalent to the known acyl-ACP thioesterases, a protein consisting of an amino acid sequence having 50% or more (preferably 70% or more, more preferably 80% or more, further preferably 90% or more, furthermore preferably 95% or more, furthermore preferably 96% or more, furthermore preferably 97% or more, furthermore preferably 98% or more, or furthermore preferably 99% or more) identity with the amino acid sequence of any one of the above-described acyl-ACP thioesterases, and having acyl-ACP thioesterase activity, can be also used.

Among the above-described acyl-ACP thioesterases, a medium chain-specific acyl-ACP thioesterase is preferable. In particular, an acyl-ACP thioesterase derived from Cocos nucifera (SEQ ID NO: 5, the nucleotide sequence of the gene encoding this thioesterase: SEQ ID NO: 6), an acyl-ACP thioesterase derived from Umbellularia californica (GenBank AAA34215.1), an acyl-ACP thioesterase derived from Cuphea lanceolata (GenBank CAA54060), an acyl-ACP thioesterase derived from Cuphea hookeriana (GenBank Q39513), and an acyl-ACP thioesterase derived from Ulumus americana (GenBank AAB71731); and a protein consisting of an amino acid sequence having 50% or more (preferably 70% or more, more preferably 80% or more, further preferably 90% or more, furthermore preferably 95% or more, furthermore preferably 96% or more, furthermore preferably 97% or more, furthermore preferably 98% or more, or furthermore preferably 99% or more) identity with the amino acid sequence of any one of these acyl-ACP thioesterases, and having medium chain-specific acyl-ACP thioesterase activity, are more preferable.

The amino acid sequence information of these acyl-ACP thioesterases, the nucleotide sequence information of the genes encoding them, and the like can be obtained from, for example, National Center for Biotechnology Information (NCBI) and the like.

The acyl-ACP thioesterase activity or the medium chain-specific acyl-ACP thioesterase activity of the protein can be measured by, for example, introducing a DNA produced by linking the acyl-ACP thioesterase gene to the downstream of a promoter which functions in a host cell such as Escherichia coli, into a host cell which lacks a fatty acid degradation system, culturing the thus-obtained cell under the conditions suitable for the expression of the introduced acyl-ACP thioesterase gene, and analyzing any change caused thereby in the fatty acid composition of the cell or the cultured liquid by using a gas chromatographic analysis or the like.

Alternatively, the acyl-ACP thioesterase activity or the medium chain-specific acyl-ACP thioesterase activity can be measured by introducing a DNA produced by linking the acyl-ACP thioesterase gene to the downstream of a promoter which functions in a host cell such as Escherichia coli, into a host cell, culturing the thus-obtained cell under the conditions suitable for the expression of the introduced acyl-ACP thioesterase gene, and subjecting a disruption liquid of the cell to a reaction which uses acyl-ACPs, as substrates, prepared according to the method of Yuan et al. (Yuan L, Voelker T A, Hawkins D J. “Modification of the substrate specificity of an acyl-acyl carrier protein thioesterase by protein engineering”, Proc. Natl. Acad. Sci. USA, 1995 Nov. 7; 92 (23), p. 10639-10643).

### 4. Transformant (Recombinant)

The transformant of the present invention can be obtained by introducing the gene encoding the protein (A) or (B), preferably the gene consisting of the DNA (a) or (b), into a host. In the transformant, in comparison with the host, the ability to produce the medium chain fatty acids, and the lipids containing as the components the medium chain fatty acids is significantly improved. Moreover, in the transformant, the fatty acid composition in the lipids is modified in comparison with the host. The ability to produce fatty acids and a lipid of the host and the transformant can be measured by the method used in Examples described below.

The transformant of the present invention can be obtained by introducing the gene encoding the protein (A) or (B), preferably the gene consisting of the DNA (a) or (b), into a host according to an ordinary genetic engineering method. The transformant of the present invention is preferably a transformant produced by introducing the gene encoding the acyl-ACP thioesterase into a host. Specifically, the transformant of the present invention can be produced by preparing an expression vector capable of expressing the gene encoding the protein (A) or (B), preferably the gene consisting of the DNA (a) or (b), in a host cell, introducing it into a host cell to transform the host cell.

A transformant having further introduced gene encoding the acyl-ACP thioesterase, preferably, the medium chain-specific acyl-ACP thioesterase can also be prepared in a similar manner.

The host for the transformant is not particularly limited, and examples of the host include microorganisms, plants or animals. In the present invention, microorganisms include algae and microalgae. Among these, microorganisms or plants are preferable, and plants are more preferable, from the viewpoints of production efficiency and usability of the obtained lipids.

As the microorganisms for the host cell, prokaryotes and eukaryotes can be used. Prokaryotes include microorganisms belonging to the genus Escherichia or microorganisms belonging to the genus Bacillus. Eukaryotes include eukaryotic microorganisms belonging to yeast or filamentous fungi. Among these, from the viewpoint of the productivity of medium chain fatty acids, Escherichia coli belonging to the genus Escherichia, Bacillus subtilis belonging to the genus Bacillus, Rhodosporidium toruloides belonging to yeast, and Mortierella sp. belonging to filamentous fungi are preferable; and Escherichia coli is more preferable.

As the microorganisms for the host cell, microalgae are also preferable. As the microalgae, from a viewpoint of establishment of a gene recombination technique, algae belonging to the genus Chlamydomonas, algae belonging to the genus Chlorella, algae belonging to the genus Phaeodactylum, and algae belonging to the genus Nannochloropsis are preferable; and algae belonging to the genus Nannochloropsis are more preferable.

As the plants for the host cell, from the viewpoint of high lipid content of seeds, Arabidopsis thaliana, rapeseed, Cocos nucifera, palm, cuphea, Amygdalus pedunculata Pall., Glycine max, Zea mays, Oryza sativa, Helianthus annuus, Cinnamomum camphora, and Jatropha curcas are preferable; and Arabidopsis thaliana is more preferable.

A vector for use as the expression vector may be any vector capable of introducing the gene encoding the protein (A) or (B), or the acyl-ACP thioesterase gene into a host cell, and expressing the gene in the host cell. For example, a vector which has expression regulation regions such as a promoter and a terminator in accordance with the type of the host cell to be used, and has a replication initiation point, a selection marker or the like, can be used. Furthermore, the vector may also be a vector such as a plasmid capable of self-proliferation and self-replication outside the chromosome, or may also be a vector which is incorporated into the chromosome.

Specific examples of the vector include, in the case of using a microorganism as the host cell, pBluescript II SK(−) (manufactured by Stratagene), a pSTV-based vector (manufactured by Takara Bio), pUC-based vector (manufactured by Takara Shuzo), a pET-based vector (manufactured by Takara Bio), a pGEX-based vector (manufactured by GE Healthcare), a pCold-based vector (manufactured by Takara Bio), pHY300PLK (manufactured by Takara Bio), pUB110 (Mckenzie, T. et al., (1986), Plasmid 15(2); p. 93-103), pBR322 (manufactured by Takara Bio), pRS403 (manufactured by Stratagene), and pMW218/219 (manufactured by Nippon Gene). In particular, in the case of using Escherichia coli as the host cell, pBluescript II SK(−) or pMW218/219 is preferably used.

When the algae are used as the host cell, specific examples of the vector include pUC19 (manufactured by Takara Bio), P66 (Chlamydomonas Center), P-322 (Chlamydomonas Center), pPha-T1 (see Yangmin Gong, Xiaojing Guo, Xia Wan, Zhuo Liang, Mulan Jiang, “Characterization of a novel thioesterase (PtTE) from Phaeodactylum tricornutum”, Journal of Basic Microbiology, 2011 December, Volume 51, p. 666-672) and pJET1 (manufactured by COSMO B10).

In the case of using a plant cell as the host cell, examples of the vector include a pRI-based vector (manufactured by Takara Bio), a pBI-based vector (manufactured by Clontech), and an IN3-based vector (manufactured by Inplanta Innovations). In particular, in the case of using Arabidopsis thaliana as the host cell, a pRI-based vector or a pBI-based vector is preferably used.

The kinds of the expression regulation regions such as a promoter and a terminator, and the selection marker are not particularly limited, and can be appropriately selected from ordinarily used promoters, markers and the like in accordance with the type of the host cell to be used.

Specific examples of the promoter include lac promoter, trp promoter, tac promoter, trc promoter, T7 promoter, SpoVG promoter, cauliflower mosaic virus 35S RNA promoter, promoters for housekeeping genes (e.g., tubulin promoter, actin promoter and ubiquitin promoter), rapeseed-derived Napin gene promoter, plant-derived Rubisco promoter, and a promoter of a violaxanthin/(chlorophyll a)-binding protein gene derived from the genus Nannochloropsis.

Examples of the selection marker include drug resistance genes such as an ampicillin resistance gene, a chloramphenicol resistance gene, an erythromycin resistance gene, a neomycin resistance gene, a kanamycin resistance gene, a spectinomycin resistance gene, a tetracycline resistance gene, a blasticidin S resistance gene, a bialaphos resistance gene, a zeocin resistance gene, a paromomycin resistance gene, and a hygromycin resistance gene. Further, it is also possible to use a deletion of an auxotrophy-related gene or the like as the selection marker gene.

The expression vector for transformation can be constructed by introducing the gene encoding the protein (A) or (B), or the acyl-ACP thioesterase gene, into the above-described vector according to an ordinary technique such as restriction enzyme treatment and ligation.

The method for transformation is not particularly limited as long as it is a method capable of introducing a target gene into a host cell. For example, a method of using calcium ion, a general competent cell transformation method (J. Bacterial. 93, 1925 (1967)), a protoplast transformation method (Mol. Gen. Genet. 168, 111 (1979)), an electroporation method (FEMS Microbiol. Lett. 55, 135 (1990)), and an LP transformation method (T. Akamatsu and J. Sekiguchi, Archives of Microbiology, 1987, 146, p. 353-357; T. Akamatsu and H. Taguchi, Bioscience, Biotechnology, and Biochemistry, 2001, 65, 4, p. 823-829) and the like, can be used. When the host is a plant, a method using Agrobacterium (C. R. Acad. Sci. Paris. Life Science 316, 1194 (1993) and the like), a particle gun method (BioRad, PDS-1000/He and the like) and the like, can be used.

The selection of a transformant having a target gene fragment introduced therein can be carried out by using the selection marker or the like. For example, the selection can be carried out by using an indicator whether a transformant acquires the drug resistance as a result of introducing a vector-derived drug resistance gene into a host cell together with a target DNA fragment. Further, the introduction of a target DNA fragment can also be confirmed by PCR using a genome as a template.

### 5. Method of Producing Medium Chain Fatty Acid

Then, the obtained transformant is used to produce a medium chain fatty acid and a lipid containing this fatty acid as a component.

The production method of the present invention contains collecting a medium chain fatty acid or a lipid containing this fatty acid as a component from the resulting transformant having the introduced gene encoding the protein (A) or (B), preferably transformant having the introduced gene encoding the protein (A) or (B) and acyl-ACP thioesterase gene. The process preferably includes a step of obtaining a cultured product by culturing, under suitable conditions, the transformant having the introduced gene encoding the protein (A) or (B), preferably transformant having the introduced gene encoding the protein (A) or (B) and acyl-ACP thioesterase gene; and a step of collecting the medium chain fatty acid or the lipid containing this fatty acid as the component from the resulting cultured product. In addition, an expression “culture the transformant” described in the present specification means culturing or growing of the microorganisms, the algae, the plants or the animals, or cells or tissues thereof, including cultivating of the plants in soil or the like. Moreover, the “cultured product” includes a transformant itself subjected to cultivation or the like, in addition to the medium used for culture.

The culture condition can be selected in accordance with the type of the host cell for transformation, and any ordinary used culture can be employed.

Further, from the viewpoint of the production efficiency of the medium chain fatty acids, precursor substances participating in the fatty acid biosynthesis, such as glycerol, acetic acid or malonic acid, may be added to the medium.

For instance, in the case of using Escherichia coli as the host cell for transformation, culture may be carried out in LB medium or Overnight Express Instant TB Medium (manufactured by Novagen) at 30° C. to 37° C. for half a day to 1 day. In the case of using Arabidopsis thaliana as the host cell for transformation, growth may be carried out at soil under the temperature conditions of 20° C. to 25° C., by continuously irradiating white light or under white light illumination conditions of a light period of 16 hours and a dark period of 8 hours, for one to two months.

When the host cell of the transformation is the algae, the following culture media and culture conditions can be applied.

As the culture medium, medium based on natural seawater or artificial seawater may be used. Alternatively, commercially available culture medium may also be used. For growth promotion of the algae and an improvement in productivity of medium chain fatty acids, a nitrogen source, a phosphorus source, a metal salt, vitamins, a trace metal or the like can be appropriately added to the culture medium. The amount of the algae to be seeded to the culture medium is not particularly limited. In view of viability, the amount per culture medium is preferably 1 to 50% (vol/vol). Culture temperature is not particularly limited within the range in which the temperature does not adversely affect growth of the algae, but is ordinarily in the range of 5° C. to 40° C. Moreover, the algae are preferably cultured under irradiation with light so that photosynthesis can be made. Moreover, the algae are preferably cultured in the presence of a carbon dioxide-containing gas or in a culture medium containing carbonate such as sodium hydrogen carbonate so that the photosynthesis can be made. In addition, the culture may be performed in any of aerated and agitated culture, shaking culture or static culture. From a viewpoint of improving air-permeability, shaking culture is preferred.

Lipids produced in the transformant is collected by an ordinary method used for isolating lipid components and the like contained in the living body of the transformant. For example, lipid components can be isolated and collected from the cultured product or the transformant by means of filtration, centrifugation, cell disruption, gel filtration chromatography, ion exchange chromatography, chloroform/methanol extraction, hexane extraction, ethanol extraction, or the like. In the case of isolation and collection of larger scales, lipids can be obtained by collecting oil components from the cultured product or the transformant through pressing or extraction, and then performing general purification processes such as degumming, deacidification, decoloration, dewaxing, and deodorization. After lipid components are isolated as such, the isolated lipids are hydrolyzed, and thereby fatty acids can be obtained. Specific examples of the method of isolating fatty acids from lipid components include a method of treating the lipid components at a high temperature of about 70° C. in an alkaline solution, a method of performing a lipase treatment, and a method of degrading the lipid components using high-pressure hot water.

The medium chain fatty acids and the lipids containing this fatty acids as the components can be efficiently produced by applying the production method of the present invention.

The lipids containing the medium chain fatty acids as the components are preferably esters of the medium chain fatty acids. Specifically, the lipids are preferably a triacylglycerol having a medium chain acyl group, or a phospholipid having a medium chain acyl group; and more preferably a triacylglycerol having a medium chain acyl group.

The medium chain fatty acids and the lipids containing these fatty acids as the components are preferably C12 to C14 fatty acids or esters thereof, more preferably C12 fatty acids or esters thereof, and particularly preferably lauric acid or an ester thereof. Higher alcohol derivatives that are obtained by reducing these higher fatty acids can be used as surfactants.

The fatty acids and lipids obtained by the production method or the transformant of the present invention can be utilized for food, as well as an emulsifier incorporated into cosmetic products or the like, a cleansing agent such as a soap or a detergent, a fiber treatment agent, a hair conditioning agent, a disinfectant or an antiseptic.

With regard to the embodiments described above, the present invention also discloses methods, transformants, proteins and genes described below.

<1> A method of producing a medium chain fatty acid or a lipid containing this fatty acid as a component, containing the steps of:

introducing a gene encoding the following protein (A) or (B) into a host, and thereby obtaining a transformant, and

collecting a medium chain fatty acid or a lipid containing this fatty acid as a component from the resulting transformant:

(A) A protein consisting of the amino acid sequence set forth in SEQ ID NO: 1; and
(B) A protein consisting of an amino acid sequence having 90% or more identity with the amino acid sequence set forth in SEQ ID NO: 1, and having medium chain acyl-ACP-specific fβ-ketoacyl-ACP synthase activity.
<2> The method of producing a medium chain fatty acid and a lipid containing this fatty acid as a component described in the above item <1>, containing the steps of:

culturing a transformant prepared by introducing the gene encoding the protein (A) or (B); and

collecting a medium chain fatty acid or a lipid containing this fatty acid as a component from the resulting cultured product.

<3> A method of modifying a fatty acid composition in a lipid, containing introducing a gene encoding the protein (A) or (B) into a host.
<4> A method of enhancing productivity of a lipid, containing introducing a gene encoding the protein (A) or (B) into a host, and thereby obtaining a transformant.
<5> The method described in any one of the above items <1> to <4>, wherein the identity in the protein (B) with the amino acid sequence set forth in SEQ ID NO: 1 is preferably 95% or more, more preferably 96% or more, more preferably 97% or more, more preferably 98% or more, and more preferably 99% or more.
<6> The method described in any one of the above items <1> to <5>, wherein the amino acid sequence of the protein (B) is an amino acid sequence in which 1 or several amino acids, preferably 1 to 10 amino acids, more preferably 1 to 5 amino acids, more preferably 1 to 3 amino acids, further preferably 1 or 2 amino acids, particularly preferably 1 amino acid, are deleted, substituted, inserted or added to the amino acid sequence set forth in SEQ ID NO: 1.
<7> The method described in any one of the above items <1> to <6>, wherein the gene encoding the protein (A) or (B) is a gene consisting of the following DNA (a) or (b):
(a) A DNA consisting of the nucleotide sequence set forth in SEQ ID NO: 2; and
(b) A DNA consisting of a nucleotide sequence having 90% or more, preferably 95% or more, more preferably 96% or more, more preferably 97% or more, more preferably 98% or more, and more preferably 99% or more, identity with the nucleotide sequence of the DNA (a), and encoding a protein having medium chain acyl-ACP-specific β-ketoacyl-ACP synthase activity.
<8> The method described in the above item <7>, wherein the identity in the DNA (b) with the nucleotide sequence set forth in SEQ ID NO: 2 is preferably 98% or more, and more preferably 99% or more.
<9> The method described in the above item <7> or <8>, wherein the nucleotide sequence of the DNA (b) is a nucleotide sequence in which 1 or several nucleotides, preferably 1 to 10 nucleotides, more preferably 1 to 5 nucleotides, more preferably 1 to 3 nucleotides, further preferably 1 or 2 nucleotides, particularly preferably 1 nucleotide, are deleted, substituted, inserted or added to the nucleotide sequence set forth in SEQ ID NO: 2.
<10> The method described in any one of the above items <1> to <9>, wherein the lipid containing the medium chain fatty acid as a component is an ester of a medium chain fatty acid.
<11> The method described in any one of the above items <1> to <10>, containing introducing a gene encoding a medium chain acyl-ACP-specific acyl-ACP thioesterase into the host.
<12> A method of producing a medium chain fatty acid or a lipid containing this fatty acid as a component, containing the steps of:

introducing a gene encoding the following protein (A) or (B1) and a gene encoding a medium chain acyl-ACP-specific acyl-ACP thioesterase into a host, and thereby obtaining a transformant, and

collecting a medium chain fatty acid or a lipid containing this fatty acid as a component from the resulting transformant:

(A) A protein consisting of the amino acid sequence set forth in SEQ ID NO: 1; and
(B1) A protein consisting of an amino acid sequence having 97% or more identity, preferably 98% or more identity, and more preferably 99% or more identity, with the amino acid sequence set forth in SEQ ID NO: 1, and having β-ketoacyl-ACP synthase activity, preferably medium chain acyl-ACP-specific β-ketoacyl-ACP synthase activity.
<13> A method of modifying a fatty acid composition in a lipid, containing introducing a gene encoding the protein (A) or (B1) and a gene encoding a medium chain acyl-ACP-specific acyl-ACP thioesterase into a host.
<14> A method of enhancing productivity of a lipid, containing introducing a gene encoding the protein (A) or (B1) and a gene encoding a medium chain acyl-ACP-specific acyl-ACP thioesterase into a host, and thereby obtaining a transformant.
<15> The method described in any one of the above items <1> to <14>, wherein the host is a microorganism or a plant.
<16> The method described in the above item <15>, wherein the plant is Arabidopsis thaliana.
<17> The method described in any one of the above items <1> to <16>, wherein the lipid contains a fatty acid having 12 carbon atoms, or an ester thereof.
<18> The protein (A) or (B).
<19> The protein described in the above item <18>, wherein the identity in the protein (B) with the amino acid sequence set forth in SEQ ID NO: 1 is preferably 95% or more, more preferably 96% or more, more preferably 97% or more, more preferably 98% or more, and more preferably 99% or more.
<20> The protein described in the above item <18> or <19>, wherein the amino acid sequence of the protein (B) is an amino acid sequence in which 1 or several amino acids, preferably 1 to 10 amino acids, more preferably 1 to 5 amino acids, more preferably 1 to 3 amino acids, further preferably 1 or 2 amino acids, particularly preferably 1 amino acid, are deleted, substituted, inserted or added to the amino acid sequence set forth in SEQ ID NO: 1.
<21> A gene encoding the protein described in any one of the above items <18> to <20>.
<22> A gene consisting of the DNA (a) or (b).
<23> The gene described in the above item <22>, wherein the identity in the DNA (b) with the nucleotide sequence set forth in SEQ ID NO: 2 is preferably 98% or more, and more preferably 99% or more.
<24> The gene described in the above item <22> or <23>, wherein the nucleotide sequence of the DNA (b) is a nucleotide sequence in which 1 or several nucleotides, preferably 1 to 10 nucleotides, more preferably 1 to 5 nucleotides, more preferably 1 to 3 nucleotides, further preferably 1 or 2 nucleotides, particularly preferably 1 nucleotide, are deleted, substituted, inserted or added to the nucleotide sequence set forth in SEQ ID NO: 2.
<25> A transformant, which is obtained by introducing the gene described in any one of the above items <21> to <24> into a host.
<26> The transformant described in the above item <25>, which is obtained by introducing a gene encoding a medium chain acyl-ACP-specific acyl-ACP thioesterase.
<27> The transformant described in the above item <25> or <26>, wherein the host is a microorganism or a plant.
<28> The transformant described in the above item <27>, wherein the plant is Arabidopsis thaliana.
<29> Use of the transformant described in any of the above items <25> to <28>, for producing a lipid.
<30> The use described in the above item <29>, wherein the lipid is a medium chain fatty acid or an ester thereof.

### EXAMPLES

Hereinafter, the present invention will be described more in detail with reference to Examples, but the present invention is not limited thereto.

The primers used in EXAMPLES are shown in Table 1.

TABLE 1

| | | SEQ ID NO: of |
|---|---|---|
| | Nucleotide sequence of primers (5′-3′) | sequence listing |
| No. 1 | gatatcactacaatgtcggagagacaaggc | SEQ ID NO: 20 |
| No. 2 | ttgtgtatgttctgtagtgatgagttttgg | SEQ ID NO: 21 |
| No. 3 | agtgtgtataccacggtgatatgagtgt | SEQ ID NO: 22 |
| No. 4 | aagctttatcggtaaaacaacgagcagag | SEQ ID NO: 23 |
| No. 5 | gggggtcgacgatatcactacaatgtcggagagacaaggctgcgcca | SEQ ID NO: 24 |
| No. 6 | gctaaagaggtggtggccatttgtgtatgttctgtagtgatgagttttggttt | SEQ ID NO: 25 |
| | gagt | |
| No. 7 | ccccccgggaagctttatcggtaaaacaacgagcagagcaagaat | SEQ ID NO: 26 |
| No. 8 | gggggtcgacgatatcactacaatgtcggagagacaaggctgcgcca | SEQ ID NO: 27 |
| No. 9 | catatgccgcggccgcccactagtttgtgtatgttctgtagtgatgagtt | SEQ ID NO: 28 |
| No. 10 | actagtgggcggccgcggcatatggtgtgtataccacggtgatatgagt | SEQ ID NO: 29 |
| No. 11 | ccccccgggaagctttatcggtaaaacaacgagcagagcaagaat | SEQ ID NO: 30 |
| No. 12 | gcggccgcatggccaccacctctttagctt | SEQ ID NO: 31 |
| No. 13 | gcggccgctctagattggtccactgcttctcagcagccg | SEQ ID NO: 32 |
| No. 14 | tggaccaatctagagctcgattccaagaagagggggg | SEQ ID NO: 33 |
| No. 15 | atatgccgcggccgctcatttactctcagttgggtgc | SEQ ID NO: 34 |
| No. 16 | ctctagattggtccactgcttctca | SEQ ID NO: 35 |
| No. 17 | gcggccgcggcatatggtgtgta | SEQ ID NO: 36 |
| No. 18 | atatatatatactagtatgagcccagaacgacg | SEQ ID NO: 37 |
| No. 19 | atatatatatcatatgatcagatctcggtgacgggca | SEQ ID NO: 38 |
| No. 20 | tgacgagttccatatggcgggactctggggttcgaa | SEQ ID NO: 39 |
| No. 21 | catcttgttcactagtgcgaaacgatccagatccggt | SEQ ID NO: 40 |
| No. 22 | taccgaggggaatttatggaacgtcagtggag | SEQ ID NO: 41 |
| No. 23 | actagtggatcctcgtgtatgtttttaatct | SEQ ID NO: 42 |
| No. 24 | actctgagattaacctatggctccccttaaa | SEQ ID NO: 43 |
| No. 25 | gaattcgtaatcatggtcatagctgtttcct | SEQ ID NO: 44 |
| No. 26 | cgaggatccactagtatggccaccacctctttagcttccgct | SEQ ID NO: 45 |
| No. 27 | catgattacgaattcaagctttatcggtaaaacaacgagc | SEQ ID NO: 46 |
| No. 28 | actagtggatcctcgtgtatgtttttaatc | SEQ ID NO: 47 |
| No. 29 | ggcatatggtgtgtataccacggtgatatg | SEQ ID NO: 48 |
| No. 30 | cgaggatccactagtatggccacaagtgctagtatcggggt | SEQ ID NO: 49 |
| No. 31 | tacacaccatatgccttaaggcatgaagggagcaaaaacaacca | SEQ ID NO: 50 |
| No. 32 | cgaggatccactagtatggccgggtactcggtggcgg | SEQ ID NO: 51 |
| No. 33 | tacacaccatatgccctatttgtaaggcgcaaacaagatag | SEQ ID NO: 52 |
| No. 34 | cgaggatccactagtatggacggcctccgccttccctccat | SEQ ID NO: 53 |
| No. 35 | tacacaccatatgcctcatggcttagaaggtgcaaatac | SEQ ID NO: 54 |

1. Extraction of RNA of Cocos nucifera

A solid endosperm derived from Cocos nucifera was frozen with liquid nitrogen and then crushed by using Multi-beads shocker (manufactured by Yasui Kikai Corporation). Phenol/chloroform and 50 mM of Tris-HCl (pH 9) were added to the crushed solid endosperm, and the resultant mixture was sufficiently mixed, subjected to centrifugation at 7,500 rpm for 10 minutes, and the resultant supernatant was collected. To the collected supernatant, similar phenol/chloroform treatment was applied again. The upper layer was collected, ethanol precipitation operation was performed thereto, and nucleic acid components contained therein were purified. In order to purify RNA components, RNeasy Plant Mini Kit (manufactured by Qiagen Inc., Valencia, Calif.) was used. A sample obtained by adding, to nucleic acid pellets after the ethanol precipitation operation, RLT Buffer to which 1/100 volume of 1M DDT was added, and vortexing the resultant mixture was applied to a QIA shredder spin column. In and after the application, operation was performed according to a manual attached to the Kit, and finally total RNA derived from Cocos nucifera was eluted with deionized water (dH₂O). To the resultant RNA solution, DNaseI (manufactured by Thermo Fisher Scientific Inc.) was added together with the Buffer, and treatment was applied thereto at 37° C. for 1 hour. Then, the phenol/chloroform treatment and the ethanol precipitation treatment were applied thereto, and the resultant solution was taken as RNA solution of the endosperm derived from Cocos nucifera.

Next, cDNA was prepared from the obtained RNA using PrimeScript II 1st strand cDNA Synthesis Kit (manufactured by Takara Bio).

2. Acyl-ACP Thioesterase Gene Derived from Cocos nucifera

A Napin gene promoter derived from Brassica raga was obtained by using a wild oilseed rape-like plant collected from Itako City, Ibaraki Prefecture, and a Napin gene terminator derived from Brassica raga was obtained by using a wild oilseed rape-like plant collected from Mashiko-cho, Tochigi Prefecture, respectively. Genome DNA of the wild oilseed rape-like plant was extracted using Power Plant DNA Isolation Kit (MO BIO Laboratories, USA). The above-described promoter and terminator were amplified by PCR using the genome DNA thus obtained as a template, and a DNA polymerase PrimeSTAR (manufactured by Takara Bio). Specifically, the Napin gene promoter derived from Brassica raga was amplified by using a pair of the primer Nos. 1 and 2, and the Napin gene terminator derived from Brassica raga was amplified by using a pair of the primer Nos. 3 and 4. Further, PCR was carried out again using the PCR products thus amplified as templates, and a pair of the primer Nos. 5 and 6 for amplifying the Napin gene promoter, or a pair of the primer Nos. 3 and 7 for amplifying the Napin gene terminator. The sequence of the Napin gene promoter is shown in SEQ ID NO: 15, and the sequence of the Napin gene terminator is shown in SEQ ID NO: 16. These amplified fragments were treated using Mighty TA-cloning Kit (manufactured by Takara Bio), subsequently the amplified fragments were respectively inserted into pMD20-T vector (manufactured by Takara Bio) by ligation. As a result, a plasmid pPNapin1 containing the Napin gene promoter and a plasmid pTNapin1 containing the Napin gene terminator were respectively constructed.

As a vector for transfection of plant cells, pRI909 (manufactured by Takara Bio) was used. The promoter and the terminator of Napin gene derived from Brassica rapa were introduced into pRI909 vector according to the following procedure. A promoter sequence to which a restriction-enzyme recognition sequence was added at both ends was amplified by PCR using PrimeSTAR with the plasmid pPNapin1 as a template, and a pair of the primer Nos. 8 and 9. Further, the terminator sequence was amplified by PCR using PrimeSTAR with the plasmid pTNapin1 as a template, and a pair of the primer Nos. 10 and 11. These amplified products were treated using Mighty TA-cloning Kit (manufactured by Takara Bio), subsequently the fragments were respectively inserted into pMD20-T vector by ligation, and thus plasmids pPNapin2 and pTNapin2 were respectively constructed. The plasmid pPNapin2 was treated with restriction enzymes SalI and NotI, and the plasmid pTNapin2 was treated with restriction enzymes SmaI and NotI. The treated plasmids were linked to pRI909 vector previously treated with restriction enzymes SalI and SmaI by ligation. Thus, plasmid p909PTnapin was constructed.

Next, a gene encoding the chloroplast transit signal peptide of the acyl-ACP thioesterase gene derived from Umbellularia californica (hereinafter, abbreviated to BTE) (SEQ ID NO: 17) was obtained utilizing customer synthesis service provided by Invitrogen (Carlsbad, Calif.). A plasmid containing a sequence of the gene obtained was used as a template, a gene fragment encoding the signal peptide was amplified by PCR using PrimeSTAR and a pair of the primer Nos. 12 and 13. The gene fragment thus amplified was treated by adding deoxyadenine (dA) to the two termini using Mighty TA-cloning Kit (manufactured by Takara Bio), subsequently the gene fragment was inserted into pMD20-T vector (manufactured by Takara Bio) by ligation. As a result, a plasmid pSignal was constructed.

The plasmid pSignal was treated with restriction enzyme NotI, and was linked to the NotI site of the plasmid p909PTnapin by ligation. Thereby, a plasmid p909PTnapin-S was obtained.

A gene sequence encoding an acyl-ACP thioesterase derived from Cocos nucifera (hereinafter, abbreviated to CTE) (SEQ ID NO: 6) was amplified by PCR using restriction enzyme PrimeSTAR MAX (manufactured by Takara Bio) with the produced cDNA of the endosperm derived from Cocos nucifera as a template, and a pair of the primer Nos. 14 and 15. Moreover, a straight chain fragment of the p909PTnapin-S was amplified by PCR using the plasmid p909PTnapin-S as a template, and a pair of the primer Nos. 16 and 17. The CTE gene fragment and the p909PTnapin-S fragment were linked by In-fusion reaction using In-Fusion Advantage PCR Cloning Kit (manufactured by Clontech), to construct a plasmid p909CTE for plant introduction. The plasmid was designed in such a manner that the CTE gene was subjected to expression control by the Napin gene promoter derived from Brassica rapa and localized to the chloroplast by the chloroplast transit signal peptide derived from the BTE gene.

3. β-ketoacyl-ACP Synthase Gene Derived from Cocos nucifera

According to the following procedure, the kanamycin resistance gene originally held by the vector pRI909 for plant introduction was substituted for the bialaphos resistance gene (Bar gene) derived from Streptomyces hygroscopicus. The Bar gene encodes a phosphinothricin acetyl transferase. The bialaphos resistance gene derived from Streptomyces hygroscopicus (SEQ ID NO: 18) was obtained utilizing customer synthesis service provided by GenScript with reference to the sequence of the vector pYW310 for transformation disclosed in Gene Bank of NCBI (ACCESSION NO. DQ469641). The Bar gene was amplified by PCR using PrimeSTAR with the artificially synthesized gene as a template, and a pair of the primer Nos. 18 and 19. Moreover, a pRI909 vector region excluding the kanamycin resistance gene was amplified by PCR using PrimeSTAR with the pRI909 as a template, and a pair of the primer Nos. 20 and 21. Both of the amplified fragments were subjected to restriction enzyme digestion with NdeI and SpeI, and then linked by ligation reaction, to construct a plasmid pRI909 Bar.

A Brassica napus Napin promoter sequence (SEQ ID NO: 19) expressed in seeds of Brassica napus was obtained utilizing customer synthesis service provided by GenScript with reference to the Brassica napus napin Promoter sequence disclosed in Gene Bank of NCBI (ACCESSION NO. EU416279). The Brassica napus Napin promoter sequence was amplified by PCR using the artificially synthesized promoter sequence as a template, and a pair of the primer Nos. 22 and 23. Moreover, a straight chain fragment of the pRI909 Bar was amplified by PCR using the plasmid pRI909 Bar as a template, and a pair of the primer Nos. 24 and 25. Moreover, a CTE-Tnapin sequence was amplified by PCR using the plasmid p909CTE as a template, and a pair of the primer Nos. 26 and 27. These amplified products were linked by In-fusion reaction in a manner similar to the method described above, to construct a plasmid p909Pnapus-CTE-Tnapin.

A straight chain fragment of the p909Pnapus-Tnapin excluding the CTE gene region was amplified by PCR using the plasmid p909Pnapus-CTE-Tnapin as a template, and a pair of the primer Nos. 28 and 29. Moreover, the CnKAS624 gene set forth in SEQ ID NO: 3 was amplified by PCR using the cDNA of the endosperm derived from Cocos nucifera as a template, and a pair of the primer Nos. 30 and 31. The obtained amplified products were linked by In-fusion reaction in a manner similar to the method described above, to construct a plasmid p909Pnapus-CnKAS624-Tnapin.

The CnKAS34 gene set forth in SEQ ID NO: 2 was amplified by PCR using the cDNA of the endosperm derived from Cocos nucifera as a template, and a pair of the primer Nos. 32 and 33, to construct a plasmid p909Pnapus-CnKAS34-Tnapin in a similar manner.

Moreover, the CnKAS1567 gene set forth in SEQ ID NO: 4 was amplified by PCR using the cDNA of the endosperm derived from Cocos nucifera as a template, and a pair of the primer Nos. 34 and 35, to construct a plasmid p909Pnapus-CnKAS1567-Tnapin in a similar manner.

The nucleotide sequence of the CnKAS624 gene set forth in SEQ ID NO: 3 has 57% identity with the nucleotide sequence of the CnKAS34 gene set forth in SEQ ID NO: 2. Moreover, the amino acid sequence encoded by the nucleotide sequence set forth in SEQ ID NO: 3 has 52% identity with the amino acid sequence set forth in SEQ ID NO: 1.

The nucleotide sequence of the CnKAS1567 gene set forth in SEQ ID NO: 4 has 58% identity with the nucleotide sequence of the CnKAS34 gene set forth in SEQ ID NO: 2. Moreover, the amino acid sequence encoded by the nucleotide sequence set forth in SEQ ID NO: 4 has 56% identity with the amino acid sequence set forth in SEQ ID NO: 1.

4. Transformation of Arabidopsis thaliana

The constructed plasmid p909CTE was supplied to the custom service for Arabidopsis thaliana transformation by Inplanta Innovations, and thus a transformant of Arabidopsis thaliana having the introduced CTE gene was obtained. The wild-type strain and the transformant of Arabidopsis thaliana were grown at room temperature of 22° C., under the conditions of a light period of 24 hours (about 4,000 lux) using fluorescent lamp illumination. After the cultivation for about 2 months, seeds were harvested.

Next, the following transformants were prepared by using the Arabidopsis thaliana transformant having the introduced p909CTE as a parental strain.

The plasmid p909Pnapus-CnKAS624-Tnapin, p909Pnapus-CnKAS1567-Tnapin and p909Pnapus-CnKAS34-Tnapin each were introduced into the Agrobacterium tumefaciens GV3101 strain, and Arabidopsis thaliana having the introduced p909CTE was transformed by using the same. To a material in which an inflorescence of Arabidopsis thaliana grown for about 1.5 months after being seeded was excised, and the Arabidopsis thaliana was further grown for six to seven days, Agrobacterium having the introduced each of the plasmids was infected. The resultant seeds were seeded to MS agar medium (containing 100 μg/mL of claforan and 7 μg/mL of bialaphos), and the transformants were selected. The obtained transformants were grown at room temperature of 22° C., under the conditions of a light period of 24 hours using fluorescent lamp illumination. After the cultivation for about 2 months, seeds were harvested.

### 5. Extraction and Methyl Esterification of Lipid

The Arabidopsis thaliana seeds thus harvested were crushed by using Multi-beads shocker (manufactured by Yasui Kikai). To the crushed seeds, 0.25 mL of chloroform containing 20 μL of 7-pentadecanone (0.5 mg/mL dissolved in methanol) (internal standard) and 20 μL of acetic acid, and 0.5 mL of methanol were added. The mixture was sufficiently stirred and then was left to stand for 15 minutes. Further, 0.25 mL of a 1.5% KCl and 0.25 mL of chloroform were added thereto, and the mixture was sufficiently stirred and then was left to stand for 15 minutes. The mixture was centrifuged for 5 minutes at room temperature and at 1,500 rpm, and then the lower layer was collected and dried with nitrogen gas. To the dried sample, 100 μL of 0.5N potassium hydroxide-methanol solution was added, and the mixture was kept at a constant temperature of 70° C. for 30 minutes to hydrolyze triacylglycerol. The dried product was dissolved by adding 0.3 mL of boron trifluoride-methanol complex solution, and the solution was kept at a constant temperature of 80° C. for 10 minutes to thereby carry out methyl esterification of fatty acids. Thereafter, 0.2 mL of saturated brine and 0.3 mL of hexane were added thereto, and the mixture was sufficiently stirred and then was left to stand for 30 minutes. The hexane layer (upper layer) containing methyl esters of fatty acids was collected and supplied to gas chromatographic (GC) analysis.

### 6. GC Analysis

The methyl-esterified samples were analyzed by GC. The GC was carried out using column: DB1-MS (J&W Scientific, Inc., Folsom, Calif.) and analysis apparatus: 6890 (Agilent Technologies, Inc., Santa Clara, Calif.), under the conditions as follows: [column oven temperature: maintained for 0.5 min. at 150° C.→150° C. to 320° C. (temperature increase at 20° C./min)→maintained for 2 min. at 320° C., injection port detector temperature: 300° C., injection method: split mode (split ratio=75:1), amount of sample injection 5 μL, column flow rate: constant at 0.3 mL/min, detector: FID, carrier gas: hydrogen, makeup gas: helium].

Amounts of the fatty acid methyl esters were quantitatively determined based on the peak areas of waveform data obtained by the above GC analysis. Meanwhile, the peak of GC corresponding to the individual lipid in the seeds was identified by the retention time (RT) of a methyl ester of a standard product of the individual fatty acid. Further, the peak area corresponding to each of the fatty acid methyl esters was compared with that of 7-pentadecanone as the internal standard, and carried out corrections between the samples, and then the amount of fatty acids contained in all the seeds supplied to analysis was calculated.

A proportion of each fatty acid contained in the total fatty acid of each Arabidopsis thaliana seed is shown in Table 2. In Table 2, for the wild strain and the strain having the introduced p909Pnapus-CnKAS1567-Tnapin gene, the value of one line was shown. For the parental strain (strain having the introduced p909CTE gene), the strain having the introduced p909Pnapus-CnKAS34-Tnapin gene and the strain having the introduced p909Pnapus-CnKAS624-Tnapin gene, the average value of three independent lines was shown. When “C18:n” was described, the description represents a total of unsaturated fatty acids having 18 carbon atoms (C18:1 to C18:3).

TABLE 2

| | | Parental | Strain having | Strain having | Strain having |
|---|---|---|---|---|---|
| | | strain | introduced | introduced | introduced |
| | | (Strain | p909Pnapus- | p909Pnapus- | p909Pnapus- |
| | | having | CnKAS34- | CnKAS624- | CnKAS1567- |
| | | introduced | Tnapin gene | Tnapin gene | Tnapin gene |
| | Wild- | p909CTE | CTE gene | CTE gene | CTE gene |
| | type | gene) | CnKAS34 | CnKAS624 | CnKAS1567 |
| | — | CTE gene | gene | gene | gene |
| C12:0 | 0.01% | 3.08% | 6.75% | 0.75% | 0.46% |
| C14:0 | 0.09% | 13.32% | 13.70% | 4.17% | 3.80% |
| C16:1 | 0.53% | 0.76% | 0.57% | 0.85% | 0.87% |
| C16:0 | 7.46% | 22.97% | 16.44% | 33.07% | 34.79% |
| C18:n | 63.03% | 39.92% | 41.71% | 40.69% | 39.99% |
| C18:0 | 2.83% | 4.39% | 3.89% | 5.22% | 4.94% |
| C20:1 | 21.77% | 9.82% | 10.61% | 9.98% | 9.74% |
| C20:0 | 1.88% | 3.63% | 3.62% | 3.51% | 3.58% |
| C22:1 | 2.09% | 1.48% | 2.06% | 1.19% | 1.23% |
| C22:0 | 0.32% | 0.63% | 0.66% | 0.57% | 0.61% |

As shown in Table 2, in the seeds of the transformant into which only the CTE gene was introduced, the proportion of the C12:0 fatty acid increased by about 3%, the proportion of the C14:0 fatty acid increased by about 13%, and the proportion of the C16:0 fatty acid increased by about 15%, in comparison with wild-type seeds.

In the seeds of the transformant into which the CTE gene and the CnKAS624 gene were introduced, and the seeds of the transformant into which the CTE gene and the CnKAS1567 gene were introduced, the proportion of the C16:0 fatty acid significantly increased, in comparison with the seeds of the transformant that expresses only the CTE gene. On the other hand, both of the proportions of the C12:0 fatty acid and the C14:0 fatty acid decreased. Both of the CnKAS624 gene and the CnKAS1567 gene were annotated as KAS I, in use of homology search by BLAST program (http://blast.ncbi.nlm.nih.gov/Blast.cgi). KAS I elongates the acyl-ACP to C16 acyl-ACP. From these results, it is considered that the CnKAS624 gene and the CnKAS1567 gene encode KAS I.

In the seeds of the transformant into which the CTE gene and the CnKAS34 gene were introduced, the proportion of the C12:0 fatty acid significantly increased, and the proportion of the C14:0 fatty acid was in a similar degree, in comparison with the seeds of the transformant that expresses only the CTE gene. On the other hand, the proportion of the C16:0 fatty acid decreased. From these results, it is considered that the CnKAS34 gene encodes a KAS IV gene having specificity to the medium chain acyl-ACP. Herein, the CnKAS34 gene was annotated as KAS II, in use of homology search by BLAST program. However, KAS II is an enzyme serving as a catalyst for a reaction of converting C16 acyl-ACP to C18 acyl-ACP, and the results are not matched with an effect of introducing the CnKAS34 gene obtained as described above. It is considered that the reason why the CnKAS34 gene was annotated as KAS II is that the KAS IV gene was hardly identified in plants.

Having described our invention as related to the present embodiments, it is our intention that the invention not be limited by any of the details of the description, unless otherwise specified, but rather be construed broadly within its spirit and scope as set out in the accompanying claims.

This application claims priority on Patent Application No. 2014-159011 filed in Japan on Aug. 4, 2014, which is entirely herein incorporated by reference.

What is claimed is:

1. A method of producing a medium chain fatty acid or a lipid containing this fatty acid as a component, comprising the steps of:
introducing a gene encoding the following protein (A) or (B) into a host, and thereby obtaining a transformant, and
collecting a medium chain fatty acid or a lipid containing this fatty acid as a component from the resulting transformant:
(A) A protein consisting of the amino acid sequence set forth in SEQ ID NO: 1; and
(B) A protein consisting of an amino acid sequence having 90% or more identity with the amino acid sequence set forth in SEQ ID NO: 1, and having medium chain acyl-ACP-specific β-ketoacyl-ACP synthase activity.

2. A method of modifying a fatty acid composition in a lipid, comprising introducing a gene encoding the following protein (A) or (B) into a host:
(A) A protein consisting of the amino acid sequence set forth in SEQ ID NO: 1; and
(B) A protein consisting of an amino acid sequence having 90% or more identity with the amino acid sequence set forth in SEQ ID NO: 1, and having medium chain acyl-ACP-specific β-ketoacyl-ACP synthase activity.

3. The method of claim 2, comprising introducing a gene encoding a medium chain acyl-ACP-specific acyl-ACP thioesterase into the host.

4. The method according to claim 1, wherein the lipid containing the medium chain fatty acid as a component is an ester of a medium chain fatty acid.

5. The method according to claim 1, comprising introducing a gene encoding a medium chain acyl-ACP-specific acyl-ACP thioesterase into the host.

6. A method of producing a medium chain fatty acid or a lipid containing this fatty acid as a component, comprising the steps of:
introducing a gene encoding the following protein (A) or (B1) and a gene encoding a medium chain acyl-ACP-specific acyl-ACP thioesterase into a host, and thereby obtaining a transformant, and
collecting a medium chain fatty acid or a lipid containing this fatty acid as a component from the resulting transformant:
(A) A protein consisting of the amino acid sequence set forth in SEQ ID NO: 1; and
(B1) A protein consisting of an amino acid sequence having 97% or more identity with the amino acid sequence set forth in SEQ ID NO: 1, and having β-ketoacyl-ACP synthase activity.

7.-8. (canceled)

9. The method according to claim 1, wherein the host is a microorganism or a plant.

10. The method according to claim 1, wherein the host is Arabidopsis thaliana.

11.-16. (canceled)

17. The method according to claim 2, wherein the host is a microorganism or a plant.

18. The method according to claim 2, wherein the host is Arabidopsis thaliana.

19. The method according to claim 6, wherein the host is a microorganism or a plant.

20. The method according to claim 6, wherein the host is Arabidopsis thaliana.
